(19) United States
(12) Patent Application Publication
(10) Pub. No.: US 2008/0170990 A1
(43) Pub. Date: Jul. 17, 2008

(54) Methods for Myocardial Imaging in Patients Having a History of Pulmonary Disease

(75) Inventors: Hsiao D. Lieu, Burlingame, CA (US); Brent Blackburn, Los Altos, CA (US); Luiz Belardinelli, Palo Alto, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CV THERAPEUTICS, INC., Palo Alto, CA (US)

(21) Appl. No.: 11/864,437

(22) Filed: Sep. 28, 2007

Related U.S. Application Data
(60) Provisional application No. 60/848,294, filed on Sep. 29, 2006.
(60) Provisional application No. 60/889,717.

(51) Int. Cl. A61K 51/04; A61K 49/00

(52) U.S. Cl. 424/1.65; 424/9.1

(57) ABSTRACT

The present application discloses methods for myocardial imaging in human patients having a history of pulmonary disease such as asthma, bronchospasm, chronic obstructive pulmonary disease, pulmonary fibrosis, pulmonary inflammation, or pulmonary hypertension, comprising administrating doses of one or more A_{2A} adenosine receptor agonists to a mammal undergoing myocardial imaging and detecting and/or diagnosing myocardial dysfunction.

This application claims priority to U.S. Provisional Patent Application Ser. No. 60/848,294, filed Sep. 29, 2007, and U.S. Provisional Patent Application Ser. No. 60/889,717, filed Feb. 13, 2007, the entirety of which are incorporated herein by reference.

### FIELD OF THE INVENTION

This invention relates to methods for myocardial imaging in human patients having a history of pulmonary disease such as asthma, bronchospasm, chronic obstructive pulmonary disease, pulmonary fibrosis, pulmonary inflammation, or pulmonary hypertension, comprising administering doses of one or more A_{2A} adenosine receptor agonists to a mammal undergoing myocardial imaging and detecting and/or diagnosing myocardial dysfunction.

### BACKGROUND

Myocardial perfusion imaging (MPI) is a diagnostic technique useful for the detection and characterization of coronary artery disease. Perfusion imaging uses materials such as radionuclides to identify areas of insufficient blood flow. In MPI, blood flow is measured at rest, and the result compared with the blood flow measured during exercise on a treadmill (cardiac stress testing), such exertion being necessary to stimulate blood flow. Unfortunately, many patients are unable to exercise at levels necessary to provide sufficient blood flow, due to medical conditions such as peripheral vascular disease, arthritis, pulmonary disorders, and the like.

Therefore, pharmacological agents that increase coronary blood flow (CBF) for a short period of time are of great benefit, particularly ones that do not cause peripheral vasodilation or act as pulmonary stress agents. Several different types of vasodilators are currently known for use in perfusion imaging. Dipyridamole is one such effective vasodilator, but side effects such as pain and nausea limit the usefulness of treatment with this compound.

Another currently marketed vasodilator is AdenoScan® (Astellas Pharma US, Inc.) which is a formulation of a naturally occurring adenosine. Adenosine (ADO), a naturally occurring nucleoside, exerts its biological effects by interacting with a family of adenosine receptors characterized as subtypes A₁, A_{2A}, A_{2B}, and A₃. Unfortunately, the use of adenosine is limited due to side effects such as flushing, chest discomfort, the urge to breathe deeply, headache, throat, neck, and jaw pain. These adverse effects of adenosine are due to the activation of other adenosine receptor subtypes in addition to A_{2A}, which mediates the vasodilatory effects of adenosine. Additionally, the short half-life of adenosine necessitates continuous infusion for 4-6 minutes during the procedure, further limiting its use.

Another side effect associated with the administration of adenosine is bronchoconstriction in asthmatic patients. Bronchoconstriction has been associated with activation of the adenosine A₃ receptors on mast cells. (See J. Linden, Trends. Pharmacol. Sci. 15: 298-306 (1994)). Furthermore, adenosine has been described as an asthma provoking agent in U.S. Pat. No. 6,248,723. Thus, the side effects of adenosine and adenosine releasing agents result substantially from non-selective stimulation of the various adenosine receptor subtypes.

Other potent and selective agonists for the A_{2A} adenosine receptor are known. For example, MRE-0470 (Medco, also known as WRC-0470 or bindodenoson) is an A_{2A} adenosine receptor agonist that is a potent and selective derivative of adenosine. This compound, which has a high affinity for the A_{2A} adenosine receptor, and, consequently, a long duration of action, has recently been shown to be useful in myocardial perfusion imaging in patients having a history of asthma or bronchospasm (U.S. published application 2006/0159621).

Thus, there is still a need for a method of producing rapid and maximal coronary vasodilation in mammals without causing corresponding peripheral vasodilation or inducing pulmonary inflammation, which would be useful for myocardial imaging with radionuclide agents. Preferred compounds would be selective for the A_{2A} adenosine receptor and have a short duration of action (although longer acting than compounds such as adenosine), thus obviating the need for continuous infusion.

### SUMMARY OF THE INVENTION

The following are aspects of this invention:

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering at least 10 μg of at least one partial A_{2A} adenosine receptor agonist to the mammal.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering no more than about 1000 μg of a partial A_{2A} adenosine receptor agonist to the mammal.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a partial A_{2A} adenosine receptor agonist in an amount ranging from about 10 to about 600 μg to the mammal.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the A_{2A} adenosine receptor is administered in a single dose.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered by iv bolus.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial wherein the partial A_{2A} adenosine receptor agonist is administered in less than about 10 seconds.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered in an amount greater than about 10 μg.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered in an amount greater than about 100 μg.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered in an amount no greater than 600 μg.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered in an amount no greater than 500 μg.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is administered in an amount ranging from about 100 μg to about 500 μg.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the partial A_{2A} adenosine receptor agonist is selected from the group consisting of CVT-3033, Regadenoson, and combinations thereof.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the myocardium is examined for areas of insufficient blood flow following administration of the radionuclide and the partial A_{2A} adenosine receptor agonist.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension comprising administering a radionuclide and a partial A_{2A} adenosine receptor agonist in an amount ranging from about 10 to about 600 μg wherein the myocardium is examined for areas of insufficient blood flow following administration of the radionuclide and the partial A_{2A} adenosine receptor agonist wherein the myocardium examination begins within about 1 minute from the time the partial A_{2A} adenosine receptor agonist is administered.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the administration of the partial A_{2A} adenosine receptor agonist causes at least a 2.5 fold increase in coronary blood flow.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the administration of the partial A_{2A} adenosine receptor agonist causes at least a 2.5 fold increase in coronary blood flow that is achieved within about 1 minute from the administration of the partial A_{2A} adenosine receptor agonist.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the radionuclide and the partial A_{2A} adenosine receptor agonist are administered separately.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the radionuclide and the partial A_{2A} adenosine receptor agonist are administered simultaneously.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the administration of the partial A_{2A} adenosine receptor agonist causes at least a 2.5 fold increase in coronary blood flow for less than about 5 minutes.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering a radionuclide and a partial A_{2A} adenosine receptor agonist in an amount ranging from about 10 to about 600 μg wherein the administration of the partial A_{2A} adenosine receptor agonist causes at least a 2.5 fold increase in coronary blood flow for less than about 3 minutes.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering Regadenoson in an amount ranging from about 10 to about 600 μg in a single iv bolus.

A method of diagnosing myocardial dysfunction during vasodilator induced myocardial stress perfusion imaging in a human patient having a history of, or diagnosis of, pulmonary disease such as, for example, asthma, bronchospasm, chronic obstructive pulmonary disease, pulmonary fibrosis, pulmonary inflammation, or pulmonary hypertension, comprising administering Regadenoson in an amount ranging from about 100 to about 500 μg in a single iv bolus.

In all of the methods above, the dose is typically administered in a single iv bolus.

In all of the methods above, at least one radionuclide is administered before, with or after the administration of the A_{2A} adenosine receptor agonist to facilitate myocardial imaging.

In all of the methods, the myocardial dysfunction includes coronary artery disease, coronary artery dilation, ventricular dysfunction, differences in blood flow through disease free coronary vessels and stenotic vessels, or a combination thereof.

In all of the methods, the method of myocardial stress perfusion imaging is a noninvasive imaging procedure. The imaging can be performed by methods including scintigraphy, single photon emission computed tomography (SPECT), positron emission tomography (PET), nuclear magnetic resonance (NMR) imaging, perfusion contrast echocardiography, digital subtraction angiography (DSA), and ultra fast X-ray computed tomography (CINE CT), and combinations of these techniques.

In certain embodiments of the method of myocardial stress perfusion imaging, the step of detecting myocardial dysfunction comprises measuring coronary blood flow velocity on the human patient to assess the vasodilatory capacity of diseased coronary vessels as compared with disease free coronary vessels.

In other embodiments of the method of myocardial stress perfusion imaging, the step of detecting myocardial dysfunction comprises assessing the vasodilatory capacity (reserve capacity) of diseased coronary vessels as compared with disease-free coronary vessels.

### DESCRIPTION OF THE FIGURES

FIG. 1 are intracoronary Doppler flow profiles following administration of 18 μg adenosine IC bolus (top) and 30 μg Regadenoson IV bolus.

FIG. 2 is a plot showing the relationship of the dose of Regadenoson on coronary peak flow rates.

FIG. 3 is a Table that reports the duration of time the coronary flow velocity is greater than or equal to 2.5 times baseline coronary flow velocity for varying doses of Regadenoson wherein “n” refers to the number of human patients dosed.

FIG. 4 is a plot of the time course of the average peak velocity (APV) ratio for human patients receiving 400 μg of Regadenoson IV bolus.

FIG. 5 is a plot of the time course of heart rate for human patients receiving 400 μg of Regadenoson IV bolus.

FIG. 6 is the time course of blood pressure for human patients receiving 400 μg of Regadenoson IV bolus.

FIG. 7 is an adverse event Table.

FIG. 8 is a plot of the change over time of mean Regadenoson plasma concentration in healthy male volunteers in a supine position. The various curves relate to different amounts of Regadenoson administered to the patients.

FIGS. 9 and 10 are plots of the mean change in heart rate of healthy male volunteers either in a standing position or in a supine position over time for various bolus dosing levels of Regadenoson.

FIG. 11 is a plot of the maximum change in heart rate in relationship to the total dose of Regadenoson administered to standing or supine human male patients. In the plot, the term “DBS” refers to the observed data point while “fit” refers to a curve fitted to the observed data points.

FIG. 12 is a plot of heart rate—(area under curve) AUC (1-15 min) of change from baseline in relationship to the total dose of Regadenoson administered to standing or supine human subjects.

FIG. 13 is a plot of the maximum change from baseline heart rate at maximum plasma concentration of Regadenoson for patients in a supine position.

FIG. 14 is a plot of heart rate—(area under the curve-time v. effect) AUCE (0-15 min) of change from baseline versus plasma AUC (0-15 min) for patients in a supine position.

FIG. 15 is a plot of the time profiles of mean heart rate change from a baseline versus mean plasma concentration over time for a 20 μg/kg dose of Regadenoson.

FIG. 16 is a plot of the average peak to blood flow velocity over time following administration of Regadenoson measured at the pulmonary artery (PA), the four limb artery (FA), brain arterial vasculature (BA) and in the left circumflex coronary artery (LCS).

FIG. 17 is a plot of the percent change in heart rate (HR) and blood pressure (BP) for various doses of Regadenoson.

FIG. 18 is a plot of the change in LBF and RBF blood flow upon administering increasing amounts of ADO or Regadenoson to awake dogs.

FIG. 19 depicts line graphs the percent change of post-bolus FEV₁ from baseline over time (minutes post bolus) for all patients during the study.

FIG. 20 depicts the average change from baseline heart rate (bpm) over time (minutes post bolus).

### DESCRIPTION OF THE INVENTION

Potent partial A_{2A} adenosine agonists are useful as adjuncts in cardiac imaging when added either prior to dosing with an imaging agent or simultaneously with an imaging agent. Suitable imaging agents include ²⁰¹Thallium or ^{99m}Technetium-Sestamibi, ^{99mTc}teboroxime, and ^{99mtc}(III).

In some embodiments of the invention, the myocardial dysfunction is detected by myocardial perfusion imaging. The imaging can be performed by methods including scintigraphy, single photon emission computed tomography (SPECT), positron emission tomography (PET), nuclear magnetic resonance (NMR) imaging, perfusion contrast echocardiography, digital subtraction angiography (DSA), and ultra fast X-ray computed tomography (CINE CT), and combinations of these techniques.

The compositions may be administered orally, intravenously (iv), through the epidermis or by any other means known in the art for administering therapeutic agents with bolus iv administration being preferred.

New and potent partial A_{2A} adenosine agonists that increase coronary blood flow (CBF) but do not significantly increase peripheral blood flow have been identified. The partial A_{2A} adenosine agonists, and especially Regadenoson and CVT-3033 have a rapid onset and a short duration when administered. An unexpected and newly identified benefit of these new compounds is that they are useful when administered in a very small quantity in a single bolus intravenous (iv) injection to human patients with a history of pulmonary disease such as asthma, bronchospasm, chronic obstructive pulmonary disease, pulmonary fibrosis, pulmonary inflammation, or pulmonary hypertension. The partial A_{2A} adenosine receptor agonists can be administered in amounts as little as 10 μg and as high as 600 μg or more and still be effective with few if any side-effects. An optimal intravenous dose will include from about 100 to about 500 μg of at least one partial A_{2A} adenosine receptor agonist. This amount is unexpectedly small when compared with adenosine which is typically administered in continuously by iv infusion at a rate of about 140 μg/kg/min. Unlike adenosine, the same dosage of partial A_{2A} adenosine receptor agonists, an in particular, Regadenoson and CVT-3033 can be administered to a human patient regardless of the patient's weight. Thus, the administration of a single uniform amount of a partial A_{2A} adenosine receptor agonist by iv bolus for myocardial imaging is dramatically simpler and less error prone than the time and weight dependent administration of adenosine.

Pharmaceutical compositions including the compounds of this invention, and/or derivatives thereof, may be formulated as solutions or lyophilized powders for parenteral administration. Powders may be reconstituted by addition of a suitable diluent or other pharmaceutically acceptable carrier prior to use. If used in liquid form the compositions of this invention are preferably incorporated into a buffered, isotonic, aqueous solution. Examples of suitable diluents are normal isotonic saline solution, standard 5% dextrose in water and buffered sodium or ammonium acetate solution. Such liquid formulations are suitable for parenteral administration, but may also be used for oral administration. It may be desirable to add excipients such as polyvinylpyrrolidinone, gelatin, hydroxy cellulose, acacia, polyethylene glycol, mannitol, sodium chloride, sodium citrate or any other excipient known to one of skill in the art to pharmaceutical compositions including compounds of this invention. Further compositions can be found in U.S. published application 2005/0020915, the specification of which is incorporated herein by reference in its entirety.

A first class of compounds that are potent and selective agonists for the A_{2A} adenosine receptor that are useful in the methods of this invention are 2-adenosine N-pyrazole compounds having the formula:

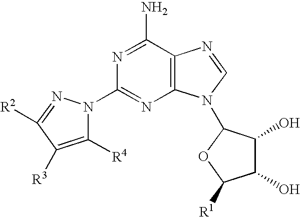

wherein

R¹═CH₂OH, —CONR⁵R⁶;

R² and R⁴ are selected from the group consisting of H, C₁₋₆ alkyl and aryl, wherein the alkyl and aryl substituents are optionally substituted with halo, CN, CF₃, OR²⁰ and N(R²⁰)₂ with the proviso that when R² is not hydrogen then R⁴ is hydrogen, and when R⁴ is not hydrogen then R² is hydrogen;

R³ is independently selected from the group consisting of C₁₋₁₅ alkyl, halo, NO₂, CF₃, CN, OR²⁰, SR²⁰, N(R²⁰)₂, S(O)R²², SO₂R²², SO₂N(R²⁰)₂, SO₂NR²COR²², SO₂NR²⁰CO₂R²², S₂NR²⁰CON(R²⁰), N(R²⁰ NR²⁰COR²², NR²⁰CO₂R²², NR²⁰CON(R²⁰)₂, NR²⁰C(NR²⁰)NHR, COR²⁰, CO₂R², CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, —CONR⁷R⁸, C₂₋₁₅ alkenyl, C₂₋₁₅ alkynyl, heterocyclyl, aryl, and heteroaryl, wherein the alkyl, alkenyl, alkynyl, aryl, heterocyclyl and heteroaryl substituents are optionally substituted with from 1 to 3 substituents independently selected from the group consisting of halo, alkyl, NO₂, heterocyclyl, aryl, heteroaryl, CF₃, CN, OR², SR²⁰, N(R²⁰)₂, S(O)R²², SO₂R², SO₂N(R²⁰)₂, SO₂NR²⁰COR²⁰, SO₂NR²⁰CO₂R²⁰, S₂NR²⁰CON(R²⁰), N(R²⁰ NR²⁰COR²², NR²⁰CO₂R²², NR²⁰CON(R²⁰)₂, NR²⁰C(NR²⁰)NHR, COR²⁰, CO₂R²⁰, CON(R²⁰)₂, CONR²SO₂R²², NR²SO₂R²², SO₂NR²⁰CO₂R²², OCONR²⁰SO₂R²², OC(O)R²⁰, C(O)OCH₂OC(O)R²⁰, and OCON(R²⁰)₂ and wherein the optional substituted heteroaryl, aryl, and heterocyclyl substituents are optionally substituted with halo, NO₂, alkyl, CF₃, amino, mono- or di-alkylamino, alkyl or aryl or heteroaryl amide, NCOR²², NR²⁰SO₂R²²COR²⁰, CO₂R², CON(R²⁰)₂, NR²⁰CON(R²⁰)₂, OC(O)R², OC(O)N(R²⁰)₂, SR², S(O)R²⁰, SO₂R²², SO₂N(R²⁰)₂, CN, or OR²⁰;

R⁵ and R⁶ are each individually selected from H, and C₁-C₁₅ alkyl that is optionally substituted with from 1 to 2 substituents independently selected from the group of halo, NO₂, heterocyclyl, aryl, heteroaryl, CF₃, CN, OR²⁰, SR²⁰, N(R²⁰)₂, S(O)R²², SO₂R²⁰, SO₂N(R²⁰)₂, SO₂NR²⁰COR²⁰, SO₂NR²⁰CO₂R²², SO₂NR²⁰CON(R²⁰)₂, N(R²⁰)₂ NR²⁰COR²², NR²⁰CO²² NR²⁰CON(R²⁰)₂, NR²⁰C(NR²⁰)NHR²³, COR²⁰, CO₂R², CON(R²⁰)₂, CONR²⁰SO₂R²², NR²⁰SO₂R²², SO₂NR²⁰CO₂R²², OCONR₂SO₂R²², OC(O)R²⁰, C(O)OCH₂OC(O)R²⁰, and OCON(R²⁰)₂ wherein each optional substituted heteroaryl, aryl, and heterocyclyl substituent is optionally substituted with halo, NO₂, alkyl, CF₃, amino, monoalkylamino, dialkylamino, alkylamide, arylamide, heteroarylamide, NCOR²², NR²⁰SO₂R²², COR²⁰, CO₂R²⁰, CON(R²⁰)₂, NR²⁰CON(R²⁰)₂, OC(O)R², OC(O)N(R²⁰)₂, SR²⁰, S(O)R²², SO²², SO₂N(R²⁰) CN, and OR²⁰;

R⁷ and R⁵ are each independently selected from the group consisting of hydrogen, C₁₋₁₅ alkyl, C₂₋₁₅ alkenyl, C₂₋₁₅ alkynyl, heterocyclyl, aryl and heteroaryl, wherein the alkyl, alkenyl, alkynyl, aryl, heterocyclyl and heteroaryl substituents are optionally substituted with from 1 to 3 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²CO²²R²²SO₂NR²⁰CON(R²⁰), N(R²⁰) NR²⁰COR²², NR²⁰CO₂R²², NR²⁰CON(R²⁰)₂, NR²⁰C(NR²⁰)NHR, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰ and OCON(R²⁰)₂ and wherein each optional substituted heteroaryl, aryl and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R², CON(R²⁰)₂, NR²⁰CON(R²⁰)₂, OC(O)R², OC(O)N(R²⁰)₂, SR², S(O)R²⁰, SO₂R²², SO₂N(R²⁰)₂, CN, and OR²⁰;

R²⁰ is selected from the group consisting of H, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl; and

R²² is selected from the group consisting of C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl.

In an related group of compounds of this invention,
R³ is selected from the group consisting of C₁₋₁₅ alkyl, halo, CF₃, CN, OR²⁰, SR²⁰, S(O)R²², SO₂R², SO₂N(R²⁰)₂, COR²⁰, CO₂R², —CONR⁷R⁸, aryl and heteroaryl wherein the alkyl, aryl and heteroaryl substituents are optionally substituted with from 1 to 3 substituents independently selected from the group consisting of halo, aryl, heteroaryl, CF₃, CN, OR²⁰, SRO, S(O)R²⁰, S₂R²⁰, SO₂N(R²⁰)₂, COR, CO₂R²⁰ or CON(R²⁰)₂, and each optional heteroaryl and aryl substituent is optionally substituted with halo, alkyl, CF₃ CN, and OR²⁰;
R⁵ and R⁶ are independently selected from the group of H and C₁-C₁₅ alkyl including one optional aryl substituent and each optional aryl substituent that is optionally substituted with halo or CF₃;
R⁷ is selected from the group consisting of C₁₋₁₅ alkyl, C₂₋₁₅ alkynyl, aryl, and heteroaryl, wherein the alkyl, alkynyl, aryl, and heteroaryl substituents are optionally substituted with from 1 to 3 substituents independently selected from the group consisting of halo, aryl, heteroaryl, CF₃, CN, OR²⁰, and each optional heteroaryl and aryl substituent is optionally substituted with halo, alkyl, CF₃ CN, or OR²⁰;
R⁸ is selected from the group consisting of hydrogen and C₁₋₁₅ alkyl;
R²⁰ is selected from the group consisting of H, C₁₋₄ alkyl and aryl, wherein alkyl and aryl substituents are optionally substituted with one alkyl substituent; and
R²² is selected from the group consisting of C₁₋₄ alkyl and aryl which are each optionally substituted with from 1 to 3 alkyl group.

In yet another related class of compounds,
R¹ is CH₂OH;
R³ is selected from the group consisting of CO₂R²⁰, —CONR⁷R⁸ and aryl where the aryl substituent is optionally substituted with from 1 to 2 substituents independently selected from the group consisting of halo, C₁₋₆ alkyl, CF₃ and OR²⁰;
R⁷ is selected from the group consisting of hydrogen, C₁₋₈ alkyl and aryl, where the alkyl and aryl substituents are optionally substituted with one substituent selected from the group consisting of halo, aryl, CF₃, CN, OR²⁰ and wherein each optional aryl substituent is optionally substituted with halo, alkyl, CF₃ CN, and OR²⁰;
R⁸ is selected from the group consisting of hydrogen and C₁₋₈ alkyl; and
R²⁰ is selected from hydrogen and C₁₋₄ alkyl.

In a still another related class of compounds of this invention,
R¹═CH₂OH;
R³ is selected from the group consisting of CO₂R²⁰, —CONR⁷R⁸, and aryl that is optionally substituted with one substituent selected from the group consisting of halo, C₁₋₃ alkyl and OR²⁰;
R⁷ is selected from of hydrogen, and C₁₋₃ alkyl;
R⁸ is hydrogen; and
R²⁰ is selected from hydrogen and C₁₋₄ alkyl.
   In this preferred embodiment, R³ is most preferably selected from —CO₂Et and —CONHEt.

In yet another related class of compounds,
R¹=—CONHEt,
R³ is selected from the group consisting of CO₂R²⁰, —CONR⁷R⁸, and aryl in that aryl is optionally substituted with from 1 to 2 substituents independently selected from the group consisting of halo, C₁₋₃ alkyl, CF₃ or OR²⁰;
R⁷ is selected from the group consisting of hydrogen, and C₁₋₈ alkyl that is optionally substituted with one substituent selected from the group consisting of halo, CF₃, CN or OR²⁰;
R⁸ is selected from the group consisting of hydrogen and C₁₋₃ alkyl; and R²⁰ is selected from the group consisting of hydrogen and C₁₋₄ alkyl.
   In this more preferred embodiment, R⁸ is preferably hydrogen, R⁷ is preferably selected from the group consisting of hydrogen, and C₁₋₃, and R²⁰ is preferably selected from the group consisting of hydrogen and C₁₋₄ alkyl.

Specific useful compounds are selected from ethyl 1-{9-[(4S,2R,3R,5R)-3,4-dihydroxy-5-(hydroxymethyl)oxolan-2-yl]-6-aminopurin-2-yl}pyrazole-4-carboxylate,
(4S,2R,3R,5R)-2-{6-amino-2-[4-(4-chlorophenyl) pyrazolyl]purin-9-yl}-5-(hydroxymethyl)oxolane-3,4-diol,
(4S,2R,3R,5R)-2-{6-amino-2-[4-(4-methoxyphenyl)pyrazolyl]purin-9-yl}-5-(hydroxymethyl)oxolane-3,4-diol,
(4S,2R,3R,5R)-2-{6-amino-2-[4-(4-methylphenyl)pyrazolyl]purin-9-yl}-5-(hydroxymethyl) oxolane-3,4-diol,
(1-{9-[(4S,2R,3R,5R)-3,4-dihydroxy-5-(hydroxymethyl)oxolan-2-yl]-6-aminopurin-2-yl}pyrazol-4-yl)-N-methylcarboxamide,
1-{9-[(4S,2R,3R,5R)-3,4-dihydroxy-5-(hydroxymethyl)oxolan-2-yl]-6-aminopurin-2-yl}pyrazole-4-carboxylic acid,
(1-{9-[(4S,2R,3R,5R)-3,4-dihydroxy-5-(hydroxymethyl)oxolan-2-yl]-6-aminopurin-2-yl}pyrazol-4-yl)-N,N-dimethylcarboxamide,
(1-{9-[(4S,2R,3R,5R)-3,4-dihydroxy-5-(hydroxymethyl)oxolan-2-yl]-6-aminopurin-2-yl}pyrazol-4-yl)-N-ethylcarboxamide,
1-{9-[(4S,2R,3R,5R)-3,4-dihydroxy-5-(hydroxymethyl)oxolan-2-yl]-6-aminopurin-2-yl}pyrazole-4-carboxamide,
1-{9-[(4S,2R,3R,5R)-3,4-dihydroxy-5-(hydroxymethyl)oxolan-2-yl]-6-aminopurin-2-yl}pyrazol-4-yl)-N-(cyclopentylmethyl)carboxamide,
(1-{9-[(4S,2R, 3R,5R)-3,4-dihydroxy-5-(hydroxymethyl)oxolan-2-yl]-6-aminopurin-2-yl}pyrazol-4-yl)-N-[(4-chlorophenyl)methyl]carboxamide,
ethyl 2-[(1-{9-[(4S,2R, 3R,5R)-3,4-dihydroxy-5-(hydroxymethyl)oxolan-2-yl]-6-aminopurin-2-yl}pyrazol-4-yl)carbonylamino]acetate, and mixtures thereof.

A second class of compounds that are potent and selective agonists for the A_{2A} adenosine receptor that are useful in the methods of this invention are 2-adenosine C-pyrazole compounds having the following formula:

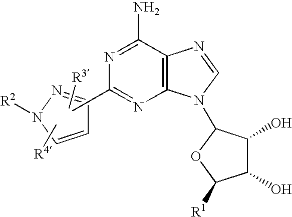

wherein

R¹ is as previously defined;

R²′ is selected from the group consisting of hydrogen, C₁₋₅ alkyl, C₂₋₁₅ alkenyl, C₂₋₁₅ alkynyl, heterocyclyl, aryl, and heteroaryl, wherein the alkyl, alkenyl, alkynyl, aryl, heterocyclyl, and heteroaryl substituents are optionally substituted with from 1 to 3 substituents independently selected from the group consisting of halo, NO₂, heterocyclyl, aryl, heteroaryl, CF₃, CN, OR²⁰, SR²⁰, N(R²⁰)₂, S(O)R²², SO₂R²², SO₂N(R²)₂, SO₂NR²⁰COR²², SON 20R²² SO₂NR²⁰ CON(R²⁰) N(R²⁰ NR²⁰COR²², NR²⁰CO₂R²², NR²⁰ CON(R²⁻⁰)₂, NR²⁰C(NR²⁰)NHR²⁰, COR²⁰, CO₂R², CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optional heteroaryl, aryl, and heterocyclyl substituent is optionally substituted with halo, NO₂, alkyl, CF₃, amino, mono-R²⁰ or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰;

R³, R⁴′ are individually selected from the group consisting of hydrogen, C₁₋₁₅ alkyl, C₂₋₁₅ alkenyl, C₂₋₁₅ alkynyl, heterocyclyl, aryl, and heteroaryl, halo, NO₂, CF₃, CN, OR²⁰, SR², N(R²⁰)₂, S(O)R²², SO₂R²², SO₂N(R²⁰)₂, SO₂NR²⁰COR²², SO₂NR²⁰C₂R²², SO₂NR²⁰CON(R²⁰) N(R²⁰)₂ NR²⁰COR²², NR²⁰CO₂R²², NR²⁰CON(R²⁰)₂, NR²⁰C(NR²⁰)NHR²³, COR²⁰, C 20 CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ wherein the alkyl, alkenyl, alkynyl, aryl, heterocyclyl, and heteroaryl substituents are optionally substituted with from 1 to 3 substituents individually selected from the group consisting of halo, NO₂, heterocyclyl, aryl, heteroaryl, CF₃, CN, OR²⁰, SR², N(R²⁰)₂, S(O)R²², SO₂R²², SO₂N(R²)₂, SO₂NR²⁰COR²², SO₂NR²², SO₂NR²⁰CON(R²⁰) N(R²⁰) NR²⁰COR²², NR²⁰CO₂R²², NR²⁰CON(R²⁰)₂, NR²⁰C(NR²⁰)NHR, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optional heteroaryl, aryl, and heterocyclyl substituent is optionally substituted with halo, NO₂, alkyl, CF₃, amino, mono-or di-alkylamino, alkyl or aryl or heteroaryl amide, NCOR²², NR²⁰SO²⁰R²², COR²⁰, CO₂R²⁰CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰; and

R⁵R⁶, R²⁰, and R²² are also as previously defined,

with the proviso that when R¹═CH₂OH, R³′ is H, R⁴′ is H, the pyrazole ring is attached through C⁴′, and R²′ is not H.

When the compound is selected has one of the following formulas:

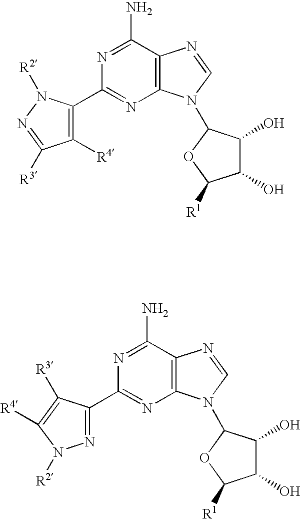

then it is preferred that R¹ is —CH₂OH; R²′ is selected from the group consisting of hydrogen, C₁₋₈ alkyl wherein the alkyl is optionally substituted with one substituent independently selected from the group consisting of aryl, CF₃, CN, and wherein each optional aryl substituent is optionally substituted with halo, alkyl, CF₃ or CN; and R³′ and R⁴′ are each independently selected from the group consisting of hydrogen, methyl and more preferably, R³′ and R⁴′ are each hydrogen.

When the compound of this invention has the following formulas:

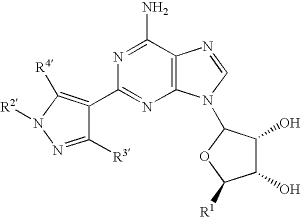

then it is preferred that R¹ is —CH₂OH; R²′ is selected from the group consisting of hydrogen, and C₁₋₆ alkyl optionally substituted by phenyl. More preferably, R²′ is selected from benzyl and pentyl; R³ is selected from the group consisting of hydrogen, C₁₋₆ alkyl, aryl, wherein the alkyl, and aryl substituents are optionally substituted with from 1 to 2 substituents independently selected from the group consisting of halo, aryl, CF₃, CN, and wherein each optional aryl substituent is optionally substituted with halo, alkyl, CF₃ or CN; and R⁴′ is selected from the group consisting of hydrogen and C₁₋₆ alkyl, and more preferably, R⁴′ is selected from hydrogen and methyl.

A more specific class of compounds is selected from the group consisting of (4S,2R,3R,5R)-2-{6-amino-2-[1-benzylpyrazol-4-yl]purin-9-yl}-5-(hydroxymethyl)oxolane-3,4-diol,
(4S,2R,3R,5R)-2-[6-amino-2-(1-pentylpyrazol-4-yl)purin-9-yl]-5-(hydroxymethyl)oxolane-3,4-diol,
(4S,2R,3R,5R)-2-[6-amino-2-(1-methylpyrazol-4-yl)purin-9-yl]-5-(hydroxymethyl)oxolane-3,4-diol,
(4S,2R,3R,5R)-2-{6-amino-2-[1-(methylethyl)pyrazol-4-yl]purin-9-yl}-5-(hydroxymethyl)oxolane-3,4-diol,
(4S,2R,3R,5R)-2-{6-amino-2-[1-(3-phenylpropyl)pyrazol-4-yl]purin-9-yl}-5-(hydroxymethyl)oxolane-3,4-diol,
(4S,2R,3R,5R)-2-{6-amino-2-[1-(4-t-butylbenzyl)pyrazol-4-yl]purin-9-yl}-5-(hydroxymethyl)oxolane-3,4-diol,
(4S,2R,3R,5R)-2-(6-amino-2-pyrazol-4-ylpurin-9-yl)-5-(hydroxymethyl)oxolane-3,4-diol,
(4S,2R,3R,5R)-2-{6-amino-2-[1-pent-4-enylpyrazol-4-yl]purin-9-yl}-5-(hydroxymethyl)oxolane-3,4-diol,
(4S,2R,3R,5R)-2-{6-amino-2-[1-decylpyrazol-4-yl]purin-9-yl}-5-(hydroxymethyl)oxolane-3,4-diol,
(4S,2R,3R,5R)-2-{6-amino-2-[1-(cyclohexylmethyl)pyrazol-4-yl]purin-9-yl}-5-(hydroxymethyl)oxolane-3,4-diol,
(4S,2R,3R,5R)-2-{6-amino-2-[1-(2-phenylethyl)pyrazol-4-yl]purin-9-yl}-5-(hydroxymethyl)oxolane-3,4-diol,
(4S,2R,3R,5R)-2-{6-amino-2-[1-(3-cyclohexylpropyl)pyrazol-4-yl]purin-9-yl}-5-(hydroxymethyl)oxolane-3,4-diol,
(4S,2R,3R,5R)-2-{6-amino-2-[1-(2-cyclohexylethyl)pyrazol-4-yl]purin-9-yl}-5-(hydroxymethyl)oxolane-3,4-diol, and combinations thereof.

A very useful and potent and selective agonists for the A_{2A} adenosine receptor is Regadenoson or (1-{9-[(4S,2R,3R,5R)-3,4-dihydroxy-5-(hydroxymethyl)oxolan-2-yl]-6-

aminopurin-2-yl}pyrazol-4-yl)-N-methylcarboxamide which has the formula:

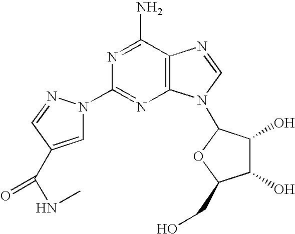

Another preferred compound that is useful as a selective partial A_{2A}-adenosine receptor agonist with a short duration of action is a compound of the formula:

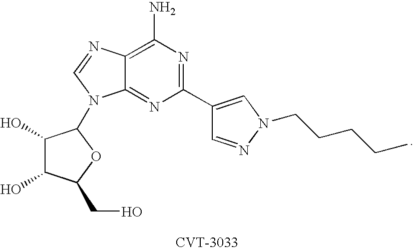

CVT-3033 is particularly useful as an adjuvant in cardiological imaging.

The first and second classes of compounds identified above are described in more detail in U.S. Pat. Nos. 6,403,567 and 6,214,807, the specification of each of which is incorporated herein by reference.

The following definitions apply to terms as used herein.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″ R″″, where R is lower alkyl, or substituted lower alkyl, R′, R′″, R″″ may independently be hydrogen, halogen, lower alkyl, substituted lower alkyl, acyl, aryl, substituted aryl, hetaryl, or substituted hetaryl as defined below.

“Alkyl alkynyl” refers to a groups —RC≡CR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” denotes the group —SR, —S(O)_{n=1-2-R}, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” denotes the group NRR′, where R and R′ may independently by hydrogen, lower alkyl, substituted lower alkyl, aryl, substituted aryl, hetaryl, or substituted hetaryl as defined herein or acyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydro furanyl, dihydropyridinyl, piperidinyl, pyrrolidinyl, piperazinyl, dihydrobenzofuryl, dihydroindolyl, and the like. A substituted heterocyclyl contains a substituent nitrogen attached at an available carbon or nitrogen to produce a stable compound.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group —R—Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group —R—HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

The first class of compounds identified above can be prepared as outlined in Schemes 1-4.

Compounds having the general formula IV can be prepared as shown in Scheme 1.

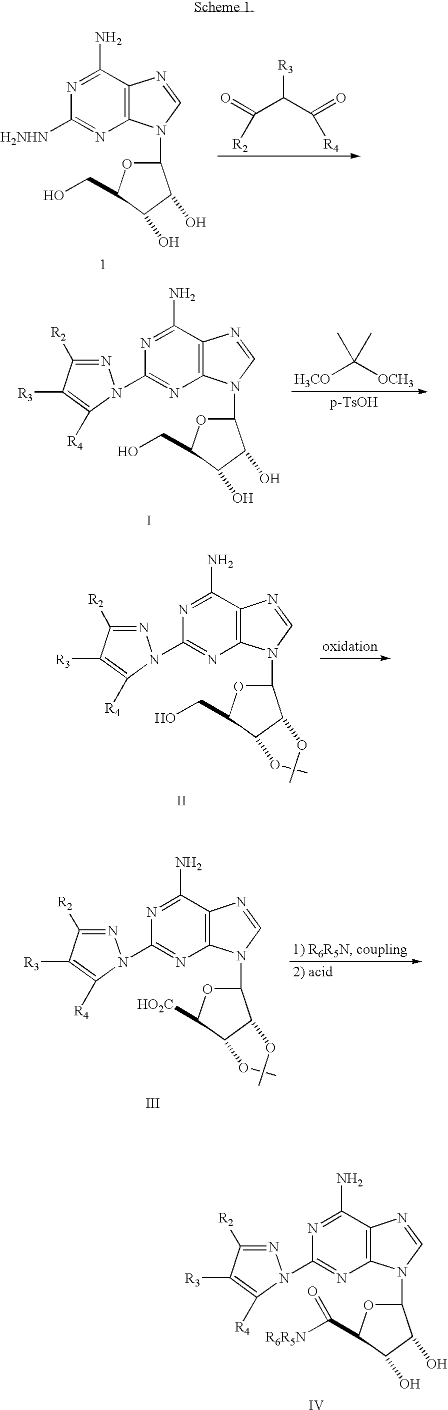

Compound I can be prepared by reacting compound 1 with appropriately substituted 1,3-dicarbonyl in a mixture of AcOH and MeOH at 80° C. (Holzer et al., J. Heterocycl. Chem. (1993) 30, 865). Compound II, which can be obtained by reacting compound I with 2,2-dimethoxypropane in the presence of an acid, can be oxidized to the carboxylic acid III, based on structurally similar compounds using potassium permanganate or pyridinium chlorochromate (M. Hudlickly, (1990) Oxidations in Organic Chemistry, ACS Monographs, American Chemical Society, Washington D.C.) Reaction of a primary or secondary amine having the formula HNR⁶R⁷, and compound III using DCC (M. Fujino et al., Chem. Pharm. Bull. (1974), 22, 1857), PyBOP (J. Martinez et al., J. Med. Chem. (1988) 28, 1874) or PyBrop (J. Caste et al. Tetrahedron, (1991), 32, 1967) coupling conditions can afford compound IV.

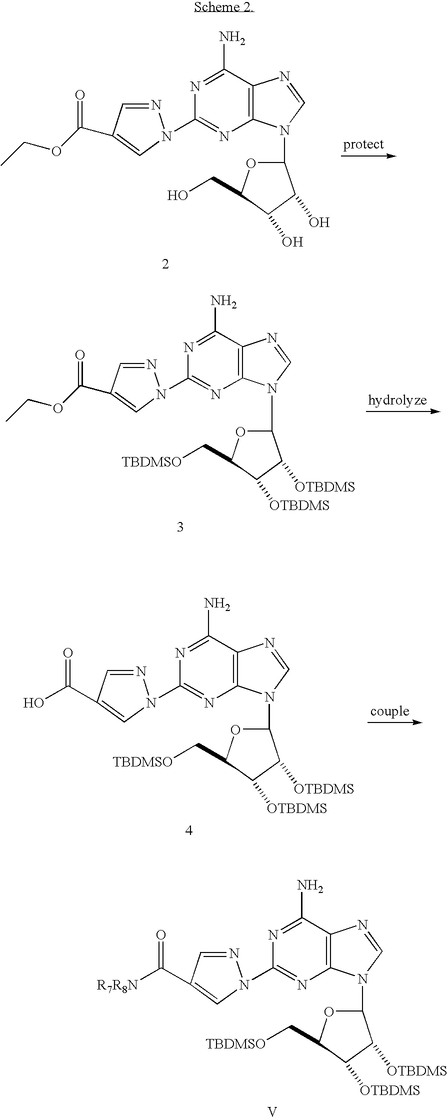

Compound V can be prepared as shown in Scheme 2. The Tri TBDMS derivative 4 can be obtained by treating compound 2 with TBDMSCl and imidazole in DMF followed by hydrolysis of the ethyl ester using NaOH. Reaction of a primary or secondary amine with the formula HNR⁶R⁷, and compound 4 using DCC (M. Fujino et al., Chem. Pharm. Bull. (1974), 22, 1857), PyBOP (J. Martinez et al., J. Med. Chem. (1988) 28, 1874) or PyBrop (J. Caste et al. Tetrahedron, (1991), 32, 1967) coupling conditions can afford compound V.

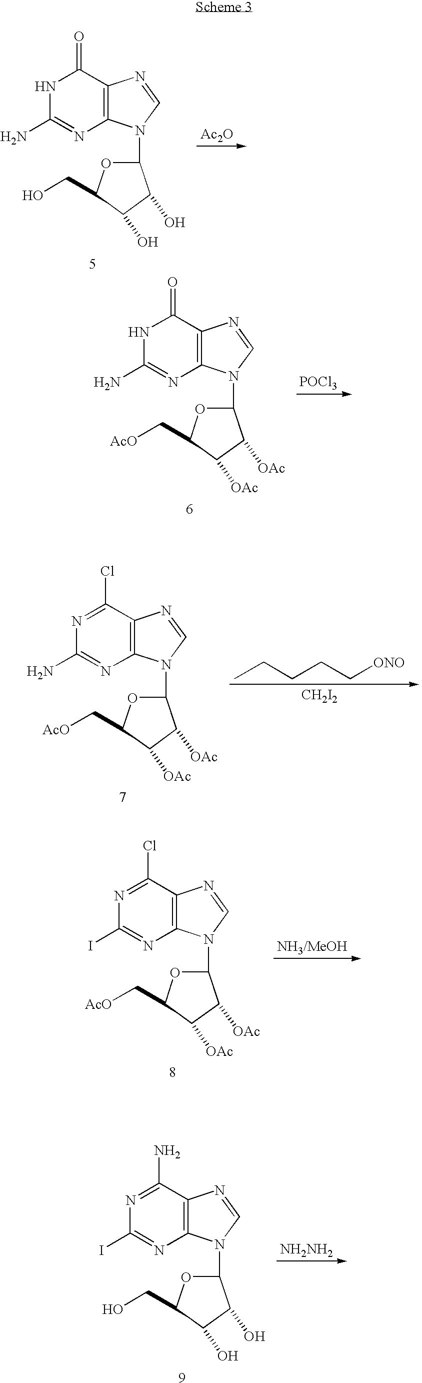

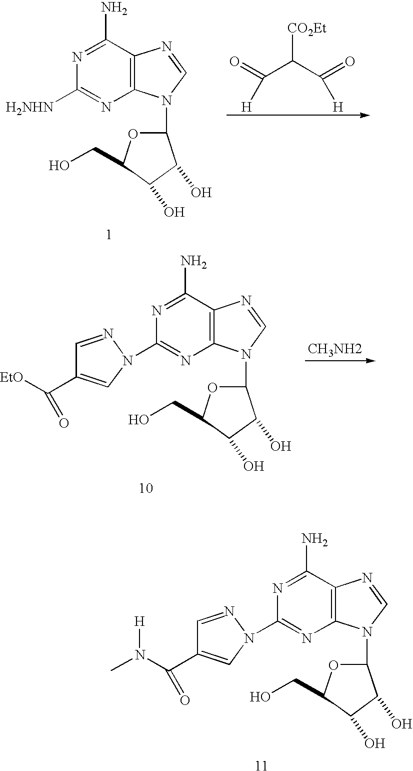

A specific synthesis of compound 11 illustrated in Scheme 3. Commercially available guanosine 5 was converted to the triacetate 6 as previously described (M. J. Robins and B. Uznanski, Can. J. Chem. (1981), 59, 2601-2607). Compound 7, prepared by following the literature procedure of Cerster et al. (J. F. Cerster, A. D. Lewis, and R. K. Robins, Org. Synthesis, 242-242), was converted to compound 9 in two steps as previously described (V. Nair et al., J. Org. Chem., (1988), 53, 3051-3057). Compound 1 was obtained by reacting hydrazine hydrate with compound 9 in ethanol at 80° C. Condensation of compound 1 with ethoxycarbonylmalondialdehyde in a mixture AcOH and MeOH at 80° C. produced compound 10. Heating compound 10 in excess methylamine afforded compound 11.

The synthesis of 1,3-dialdehyde VII is described in Scheme 4.

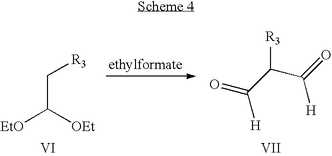

Reaction of 3,3-diethoxypropionitrile or 1,1-diethoxy-2-nitroethane VI (R₃═CO₂R, CN or NO₂) with ethyl or methyl formate in the presence of NaH can afford the dialdehyde VII (Y. Yamamoto et al., J. Org. Chem. (1989) 54, 4734).

The second class of compound described above may be prepared by as outlined in Schemes 5-9. As shown in Scheme 5, compounds having the general formula VIII:

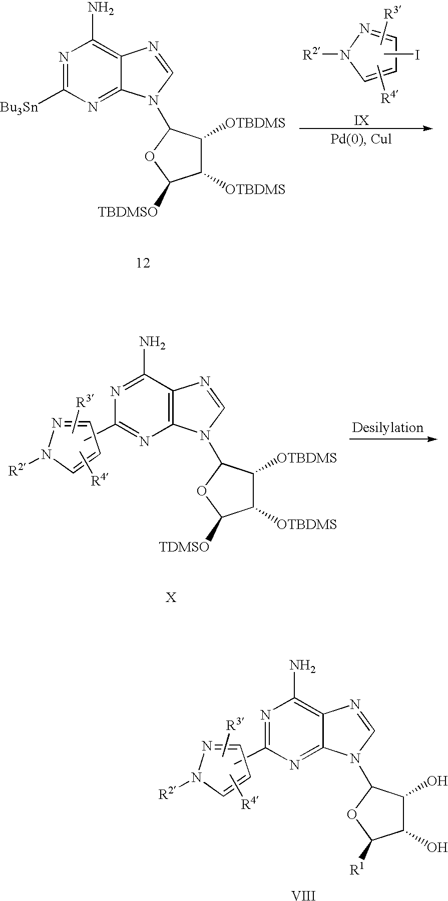

were prepared by the palladium medicated coupling of compound 12 with halo-pyrazoles represented by the formula IX (synthesis shown in Scheme 8) in the presence or absence of copper salts (K. Kato et al. J. Org. Chem. 62, 6833-6841; Palladium Reagents and Catalysts-Innovations in Organic Synthesis, Tsuji, John Wiley and Sons, 1995) followed by de-protection with either TBAF or NH₄F (Markiewicz et. al Tetrahedron Lett. (1988), 29, 1561). The preparation of compound 12 has been previously described (K. Kato et. al. J. Org. Chem. 1997, 62, 6833-6841) and is outlined in Scheme 11.

Compounds with general formula XIV can be prepared as shown in Scheme 6.

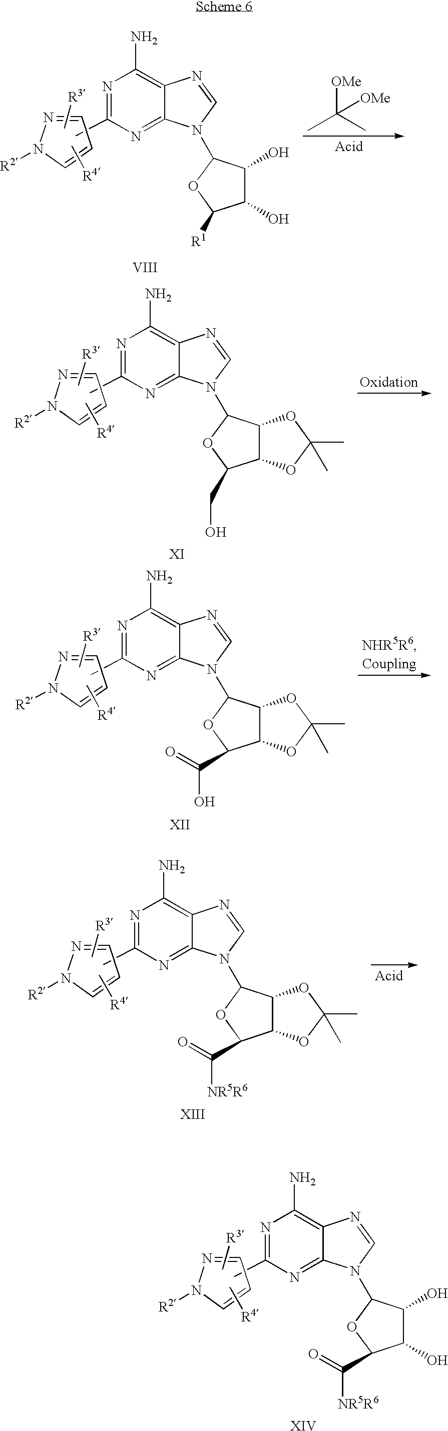

Compound IX, which can be obtained by reacting VII with 2,2-dimethoxypropane in presence of an acid, can be oxidized to the carboxylic acid XII, based on structurally similar compounds, using potassium permanganate or pyridinium chlorochromate etc. (Jones et. al., J. Am. Chem. Soc. (1949), 71, 3994; Hudlickly, Oxidations in organic chemistry, American Chemical Society, Washington D.C., 1990).

Reaction of a primary or secondary amine of the formula NHR⁵R⁶, and compound XII using DCC (Fujino et. al., Chem. Pharm. Bull. (1974), 22, 1857), PyBOP (J. Martinez et. al., J. Med. Chem. (1988), 28, 1967) or PyBrop (J. Caste et. al. Tetrahedron, (1991), 32, 1967) coupling conditions can afford compound XIII.

Deprotected of compound XIII can be performed by heating with 80% aq. acetic acid (T. W. Green and P. G. M. Wuts, (1991), Protective Groups in Organic Synthesis, A, Wiley-Interscience publications) or with anhydrous HCl (4N) to obtain compound of the general formula XIII.

Alternatively, compounds with the general formula VIII can also be prepared by Suzuki type coupling as shown in Scheme 7.

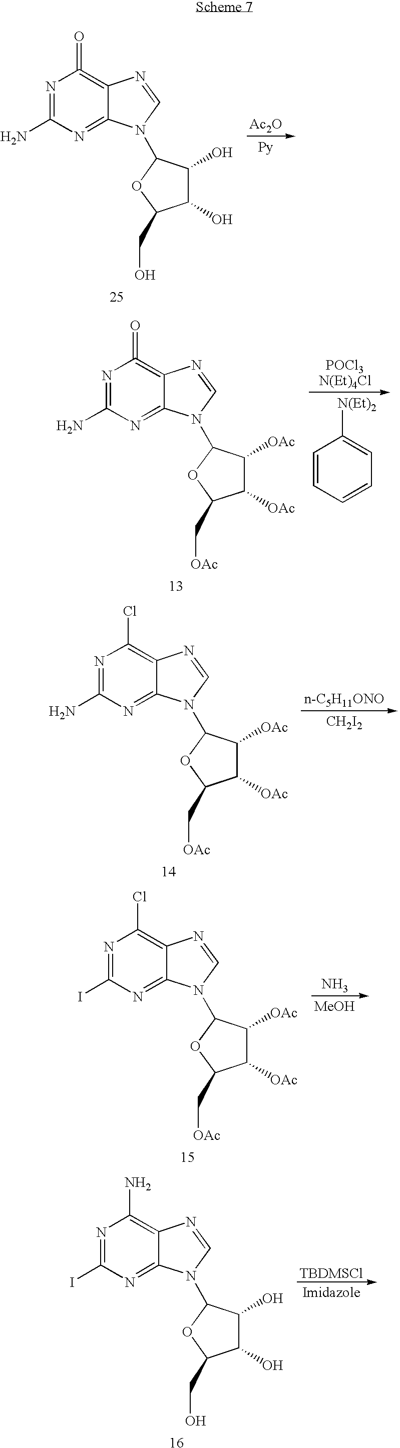

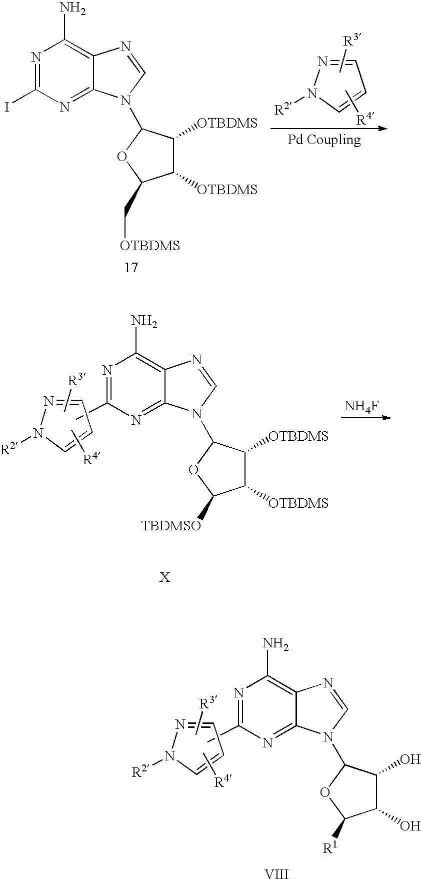

2-Iodoadenosine 16 can be prepared in four steps from guanosine 25 following literature procedures (M. J. Robins et. al. Can. J. Chem. (1981), 59, 2601-2607; J. F. Cerster et. al. Org. Synthesis,—242-243; V. Nair at. al., J. Org. Chem., (1988), 53, 3051-3057). Palladium mediated Suzuki coupling of 16 with appropriately substituted pyrazole-boronic acids in presence of a base can provide final compounds with general formula VIII (A. Suzuki, Acc. Chem Res) (1982), 15, 178). If necessary, 2′, 3′, 5′ hydroxyls on 6 can be protected as TBDMS ethers prior to Suzuki coupling.

Compounds with the general formula IX can be either commercially available or prepared following the steps shown in Scheme 8.

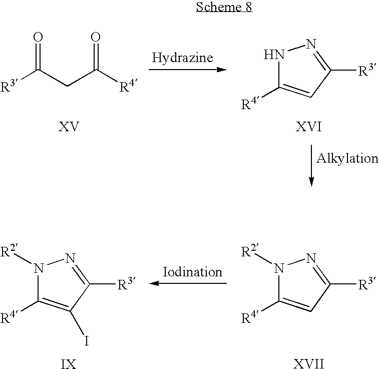

Condensation of 1,3-diketo compounds of the formula XV with hydrazine in an appropriate solvent can give pyrazoles with the general formula XVI (R. H. Wiley et. al., Org. Synthsis, Coll. Vol IV (1963), 351. These pyrazoles can be N-alkylated with various alkyl halides to give compounds of the formula XVII which on iodination give 4-iodo derivatives with the general formula IX (R. Huttel et. al. Justus Liebigs Ann. Chem. (1955), 593, 200).

5-iodopyrazoles with the general formula XXI can be prepared following the steps outlined in Scheme 9.

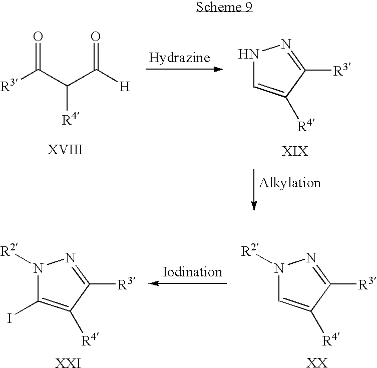

Condensation of 1,2-diketo compounds of the formula XVIII with hydrazine in an appropriate solvent can give pyrazoles with the general formula XIX. These pyrazoles can be N-pounds alkylated with various alkyl halides to give compounds of the formula XX. Abstraction of 5-H with a strong base followed by quenching with iodine can provide 5-iodo derivatives with general formula XXI (F. Effenberger et al. J. Org. Chem. (1984), 49, 4687).

4- or 5-iodopyrazoles can be transformed into corresponding boronic acids as shown in the Scheme 10.

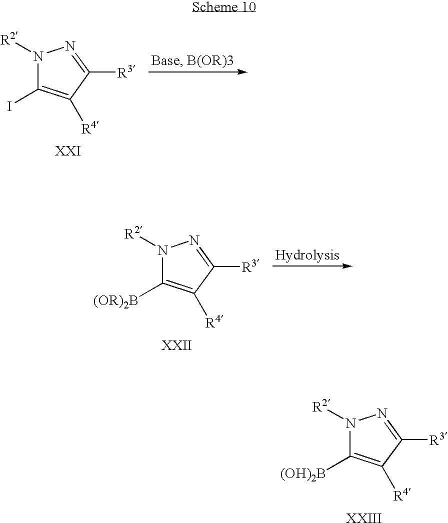

Transmetallation with n-buLi followed by treatment with trimethylborate can give compounds with the general formula XXII which on hydrolysis can provide boronic acids with the general formula XXIII (F. C. Fischer et al. RECUEIL (1965), 84, 439).

As shown in Scheme 11 below, 2-Stannyladenosine 12 was prepared in three steps from the commercially available 6-chloropurine riboside following literature procedure (K. Kato et. al., J. Org. Chem. (1997), 62, 6833-6841).

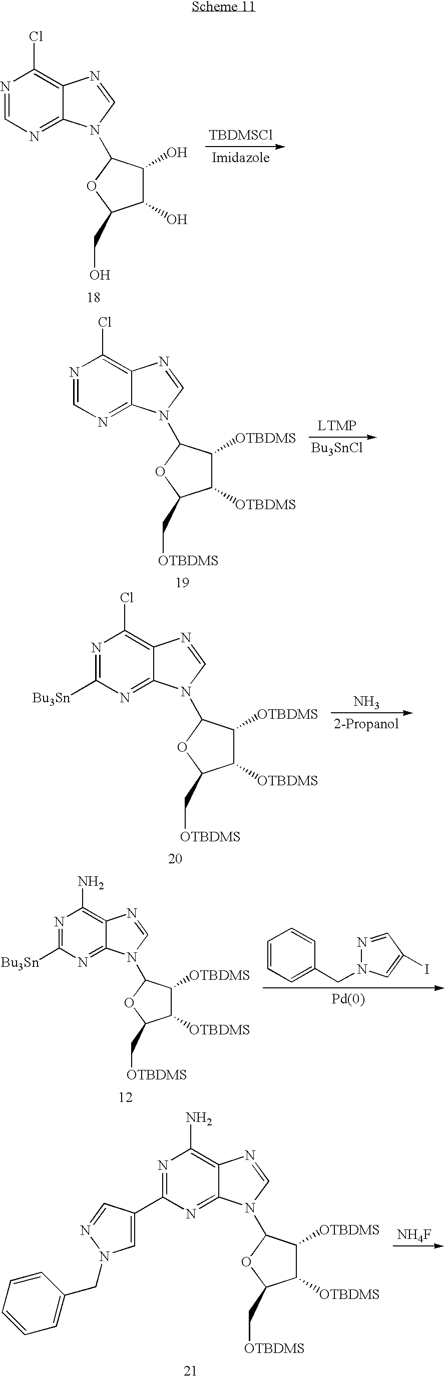

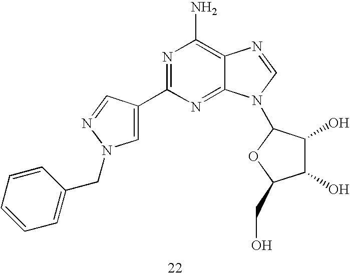

Tri TBDMS derivation was obtained by treating 18 with TBDMSCl and imidazole in DMF. Lithiation with LTMP followed by quenching with tri n-butyltin chloride gave exclusively 2-stannyl derivation 20. Ammonolysis in 2-propanol gave 2-stannyladenosine 12. Stille coupling of 12 with 1-benzyl-4-iodopyrazole in presence of Pd(PPh₃)₄ and CuI resulted in 21 (K. Kato et al. J. Org. Chem. (1997), 62, 6833-6841). Deprotection of silyl groups on 2′,3′ and 5′ hydroxyls with 0.5 M ammonium fluoride in methanol gave 22 in good yield.

The methods used to prepare the compounds of this invention are not limited to those described above. Additional methods can be found in the following sources and are included by reference (J. March, Advanced Organic Chemistry; Reaction Mechanisms and Studies (1992), A Wiley Interscience Publications; and J. Tsuji, Palladium reagents and catalysts-Innovations in organic synthesis, John Wiley and Sons, 1995).

If the final compound of this invention contains a basic group, an acid addition salt may be prepared. Acid addition salts of the compounds are prepared in a standard manner in a suitable solvent from the parent compound and an excess of acid, such as hydrochloric, hydrobromic, sulfuric, phosphoric, acetic, maleic, succinic, or methane sulfonic. The hydrochloric salt form is especially useful. If the final compound contains an acidic group, cationic salts may be prepared. Typically the parent compound is treated with an excess of an alkaline reagent, such as hydroxide, carbonate or alkoxide, containing the appropriate cation. Cations such as Na⁺, K⁺, CaW⁺² and NH₄⁺ are examples of cations present in pharmaceutically acceptable salts. Certain of the compounds form inner salts or zwittcrions which may also be acceptable.

The invention now having been fully described, it will be apparent to one of ordinary skill in the art that many changes and modifications can be made thereto without departing from the spirit or scope of the invention.

### EXAMPLE 1

### Background

Regadenoson (CV Therapeutics), with an initial half-life of 3 minutes with a rapid onset and offset of action, is >100-fold more potent than adenosine (Ado) in increasing coronary blood flow velocity (CBFv) in awake dogs. The purpose of this open label study was to determine the magnitude and duration of effect of Regadenoson (10-500 μg) on CBFv in humans.

### Methods:

Patients undergoing a clinically indicated coronary catheterization with no more than a 70% stenosis in any coronary artery and no more than a 50% stenosis of the study vessel had CBFv determined by Doppler flow wire. Study subjects were selected after measuring baseline and peak CBFv after an intracoronary (IC) injection of 18 μg of Ado. Twenty-three patients, who were identified as meeting the study criteria of having a peak to baseline CBFv ration of ≧2.5 in response to Adenosine, received a rapid (≦10 sec) peripheral IV bolus of Regadenoson; Doppler signals were stable and interpretable over the time-course of the increase in CBFv in 17 patients.

### Results

Regadenoson caused a rapid increase in CBFv that was near peak by 30 to 40 seconds post onset of bolus. Regadenoson at doses of 100 μg (n=3), 300 μg (n=4), and 500 μg (n=2) induced a peak to baseline ratio of 3.2±0.6 (mean ±SD), similar to that obtained by IC Ado (3.2±0.5). The duration of CBFv augmentation (≧2-fold increase in CBFv) was dose dependent; at 300 μg the duration was 4.0±4.9 minutes and at 500 μg was 6.9±7.6 minutes. At 500 μg (n=3) the maximal increase in HR was 18.7±4.0 and the maximal decrease in systolic BP was 8.7±7.6. Adverse events (AEs) were infrequent and included nausea, flushing, and headache; these were mild and self-limited. No AEs were noted in the 3 patients receiving the 500 μg dose.

### Conclusion

In humans peak CBFv following Regadenoson (IV bolus) is comparable to CBFv following IC Ado without major changes in either HR or BP. This agent's magnitude and duration of effect, adverse event profile and bolus administration make Regadenoson a useful pharmacological stress agent for myocardial perfusion imaging.

### EXAMPLE 2

This example is a study performed to determine the range of dosages over which the selective A_{2A} adenosine receptor agonist, Regadenoson can be administered and be effective as a coronary vasodilator.

The study included patients undergoing a clinically indicated coronary catheterization with no more than a 70% stenosis in any coronary artery and no more than a 50% stenosis of the study vessel had CBFv determined by Doppler flow wire. Study subject were selected after measuring baseline and peak CBFv after an intracoronary (IC) injection of 18 μg of Ado. 36 subjects were identified as meeting the study criteria of having a peak to baseline CBFv ration of ≧2.5 in response to Adenosine,

Regadenoson was administered to the study subjects by IV bolus in less than 10 seconds in amounts ranging from 10 μg to 500 μg. Regadenoson is selective for the A_{2A} adenosine receptor.

The effectiveness of both compounds was measured by monitoring coronary flow velocity. Other coronary parameters that were monitored included heart rate and blood pressure. These parameters were measured in order to evaluate the time to peak dose response, the magnitude of the dose response and the duration of the dose response. Adverse events were also monitored. Coronary blood flow velocity was measured at the LAD or LCx vessel. The velocity measurements were taken by following standard heart catheterization techniques and inserting a 0.014 inch Doppler-tipped Flowire into the LAD or LCx vessel and thereafter monitoring blood flow velocity. In addition, hemodynamic and electrocardiographic measurements were recorded continuously.

Overall, 36 human subjects (n=36) were evaluated. Of the 36, 18 were female and 18 were male. Their mean age was 53.4 and they ranged from 24-72 years in age. Of the 36 subjects evaluated, the LAD vessel of 31 subjects was monitored, and the LCx vessel of 5 subjects was monitored. The following doses (μg) of Regadenoson were given to the subjects in a single iv bolus: 10 (n=4); 30 (n=6); 100 (n=4); 300 (n=7); 400 (n=9); 500 (n=6).

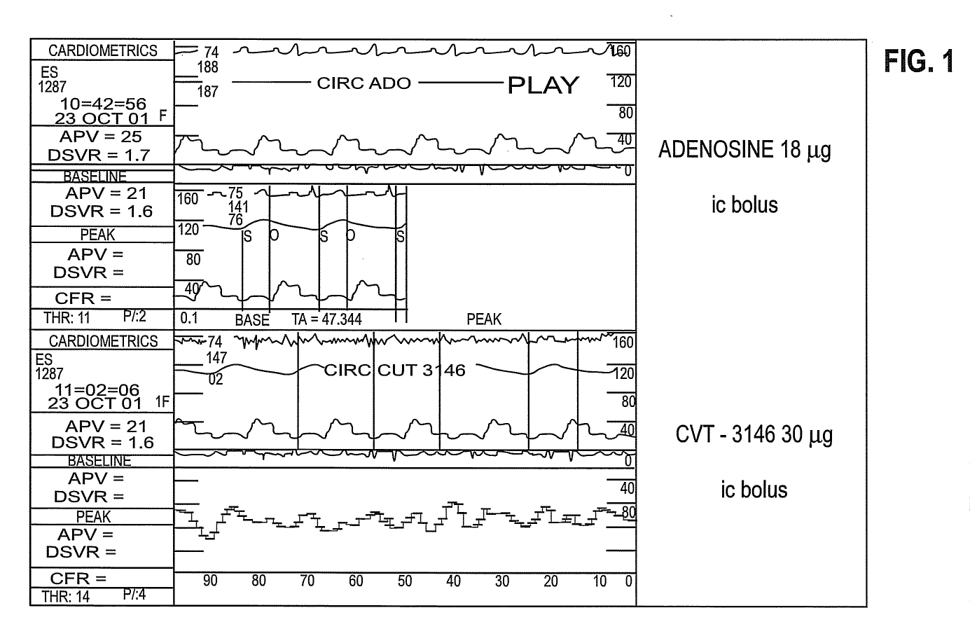

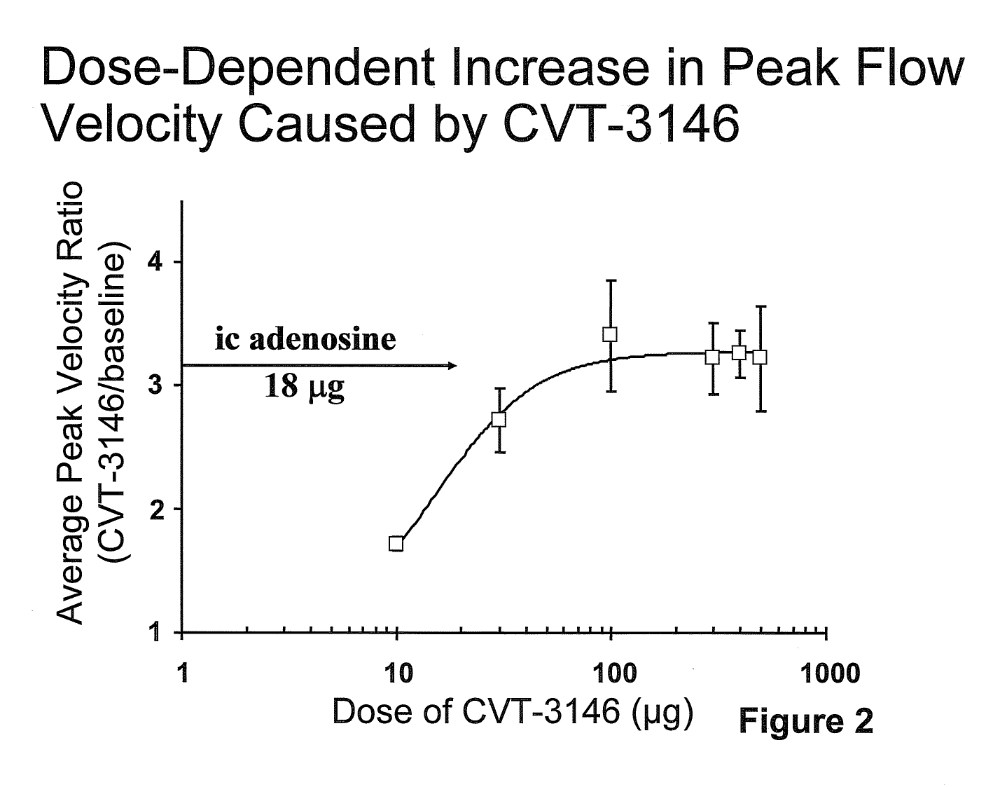

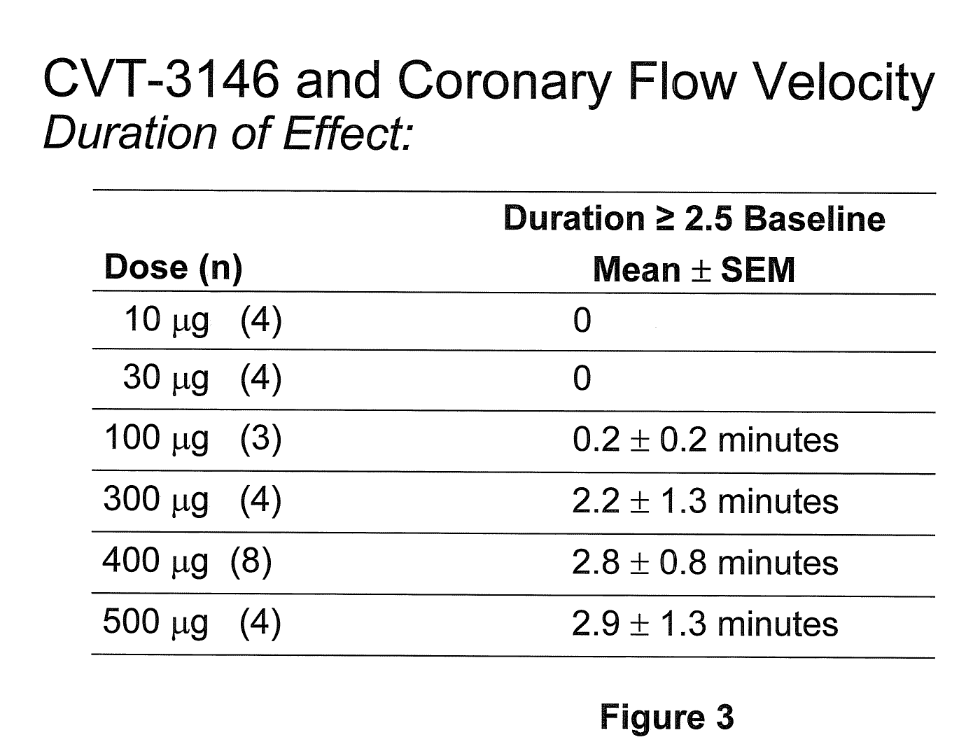

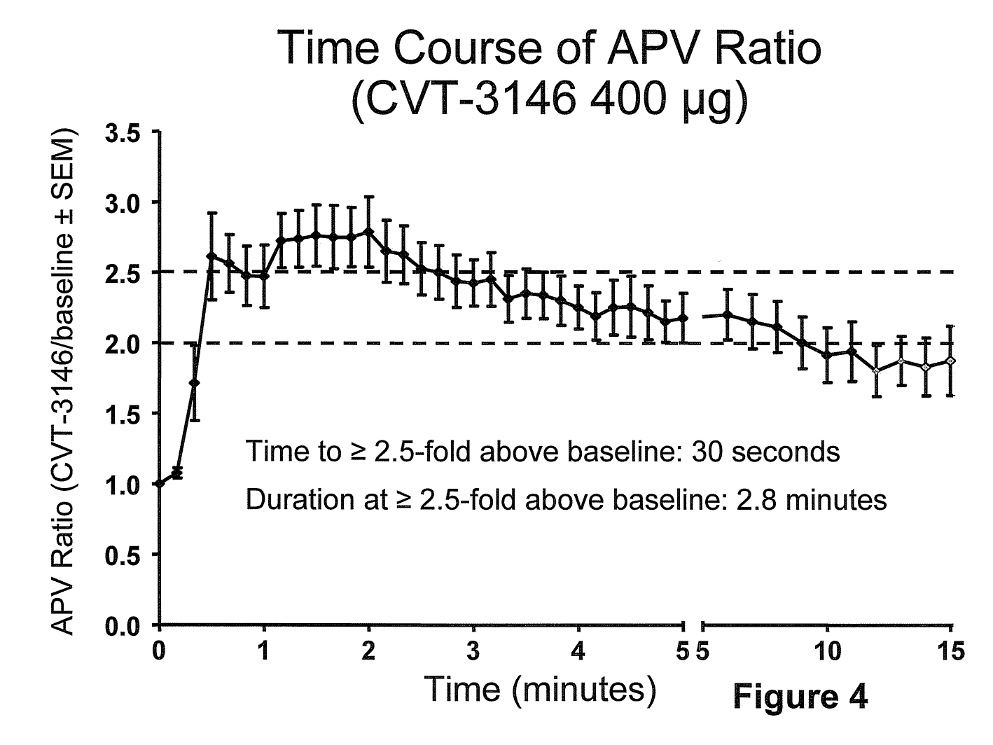

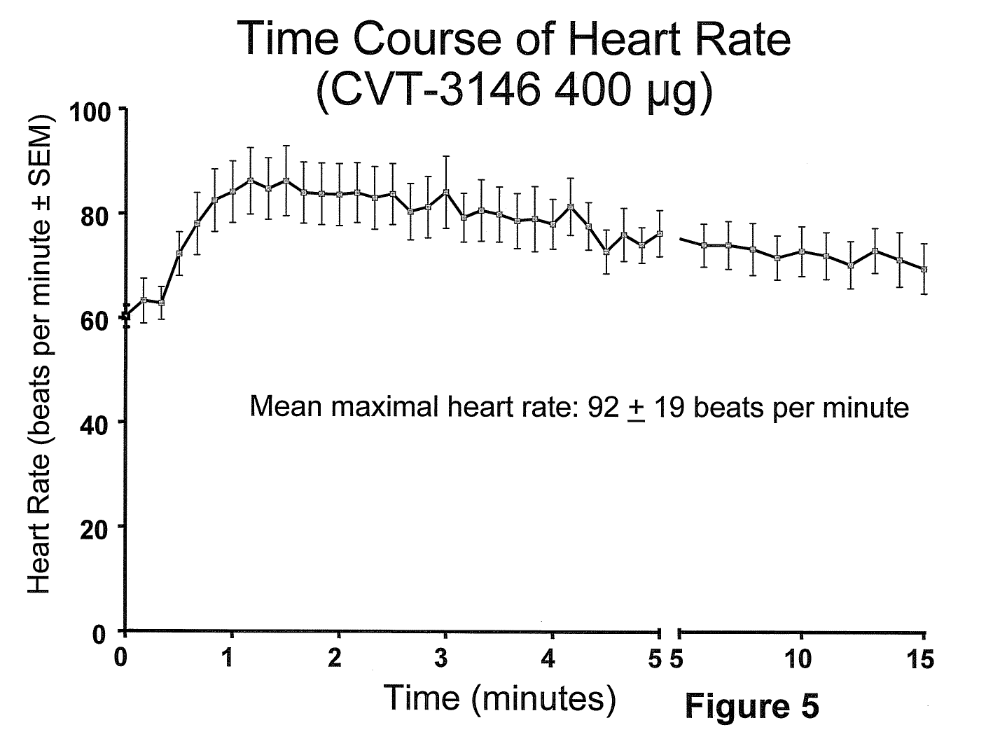

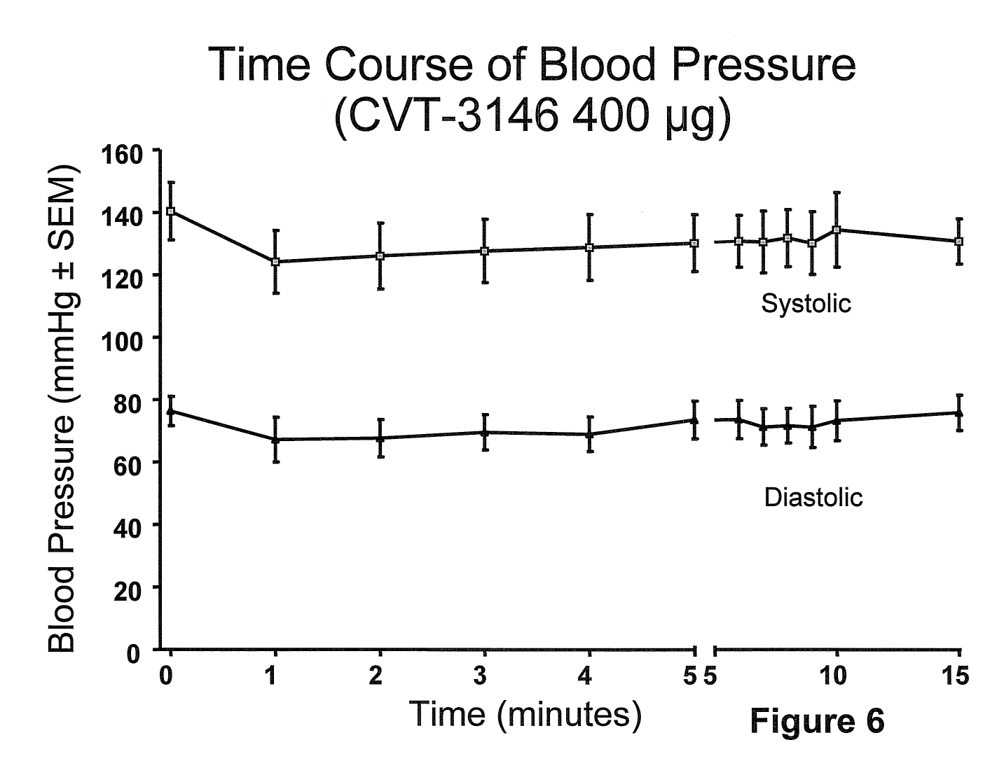

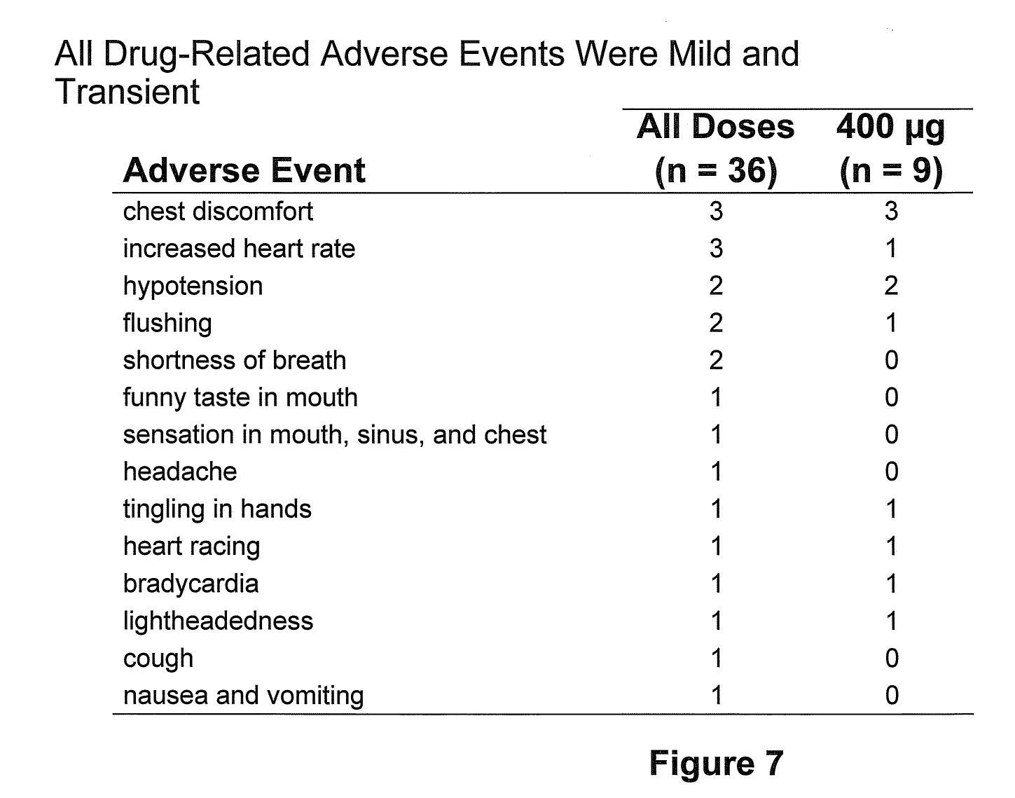

The study results are reported in FIGS. 1-6. The plot of FIG. 1 shows that Regadenoson increases peak flow velocity in amounts as low as 10 μg and reaches plateau peak velocity upon administration of less than about 100 μg of Regadenoson. Other test results and conclusions include:
The peak flow was reached by about 30 seconds with all doses;
At does above about 100 μg, peak effects were equivalent to 18 μg ic adenosine;
Regadenoson was generally well tolerated with adverse events being reported in The table attached as FIG. 7;
At 400 μg:
   Coronary blood flow velocity ≧2.5-fold above baseline was maintained for 2.8 minutes.
   Maximum increase in heart rate (18±8 bpm) occurs about 1 minute after dosing.
   Maximum decrease in systolic BP (20±8 mmHg) occurs about 1 minute after dosing.
   Maximum decrease in diastolic BP (10±5 mmHg) occurs about 1 minute after dosing.

### EXAMPLE 3

This Example is a study performed to evaluate (1) the maximum tolerated dose of Regadenoson and (2) the pharmacokinetic profile of Regadenoson in healthy volunteers, after a single IV bolus dose.

### Methods

The study was performed using thirty-six healthy, non-smoking male subjects between the ages of 18 and 59 and within 15% of ideal body weight.

### Study Design

The study was performed in phase 1, single center, double-blind, randomized, placebo-controlled, crossover, ascending dose study. Randomization was to Regadenoson or placebo, in both supine and standing positions.

Regadenoson was administered as an IV bolus (20 seconds) in ascending doses of 0.1, 0.3, 1.3, 10, 20 and 30 μg/kg.

Subjects received either Regadenoson of placebo on Day 1 supine, then crossover treatment on Day 2 supine. On Day 3, subjects received Regadenoson or placebo standing, then crossover treatment on Day 4 standing.

### Assessments

Patient safety was monitored by ECG, laboratory assessments, and collection of vital signs and adverse events.

### Pharmacokinetics:

Plasma samples were drawn during supine phase (Days 1 and 2) at 0, 1, 2, 3, 4, 5, 7, 10, 15, 20, 30, 45 minutes after dosing and at 1, 1.5, 2, 4, 6, 8, 12 and 24 hours after dosing. Urine was collected for 24 hours for Regadenoson excretion.

### Pharmacodynamics:

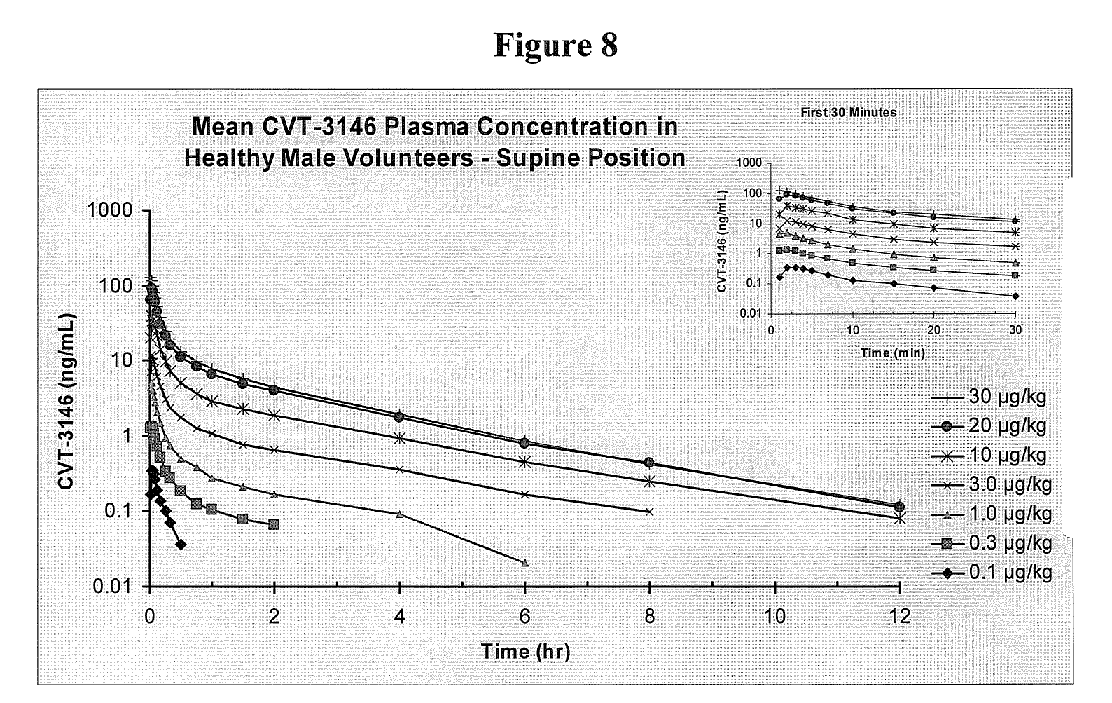

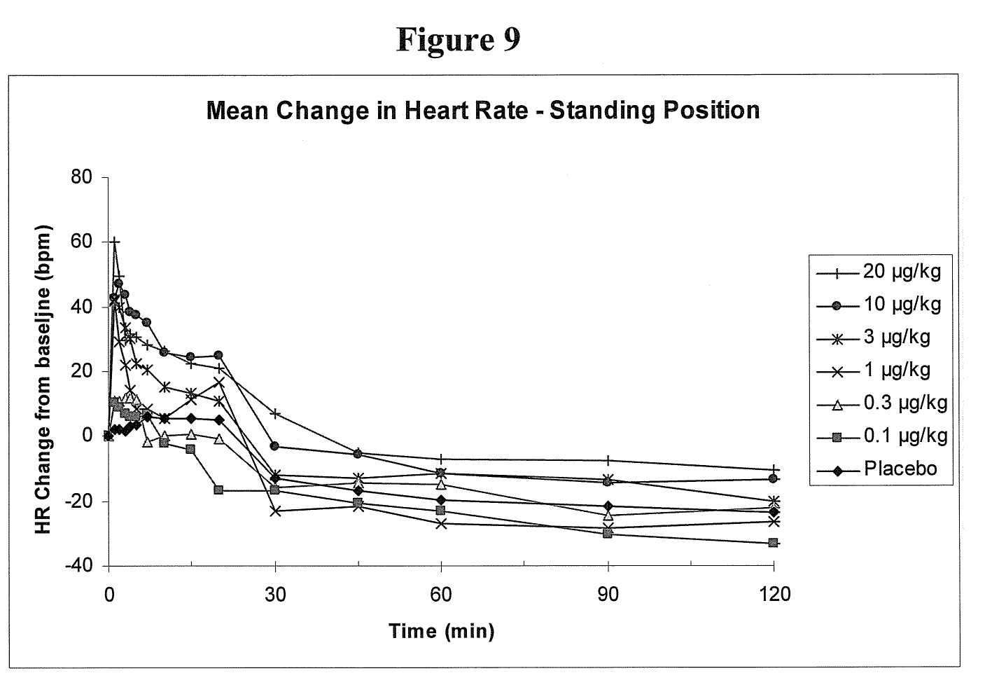

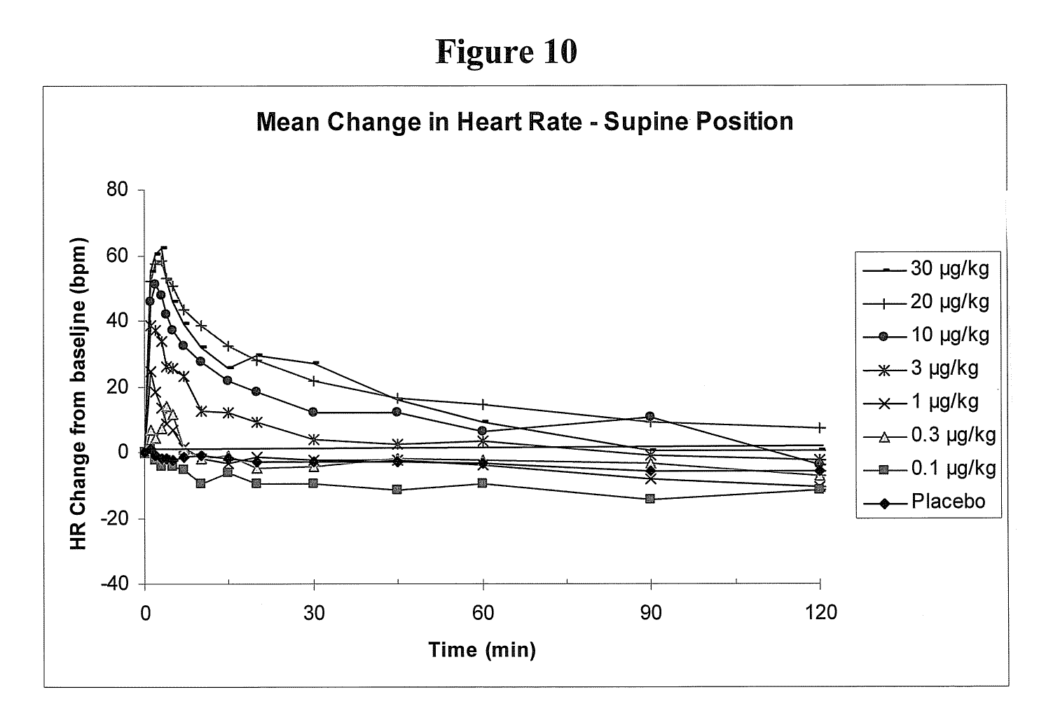

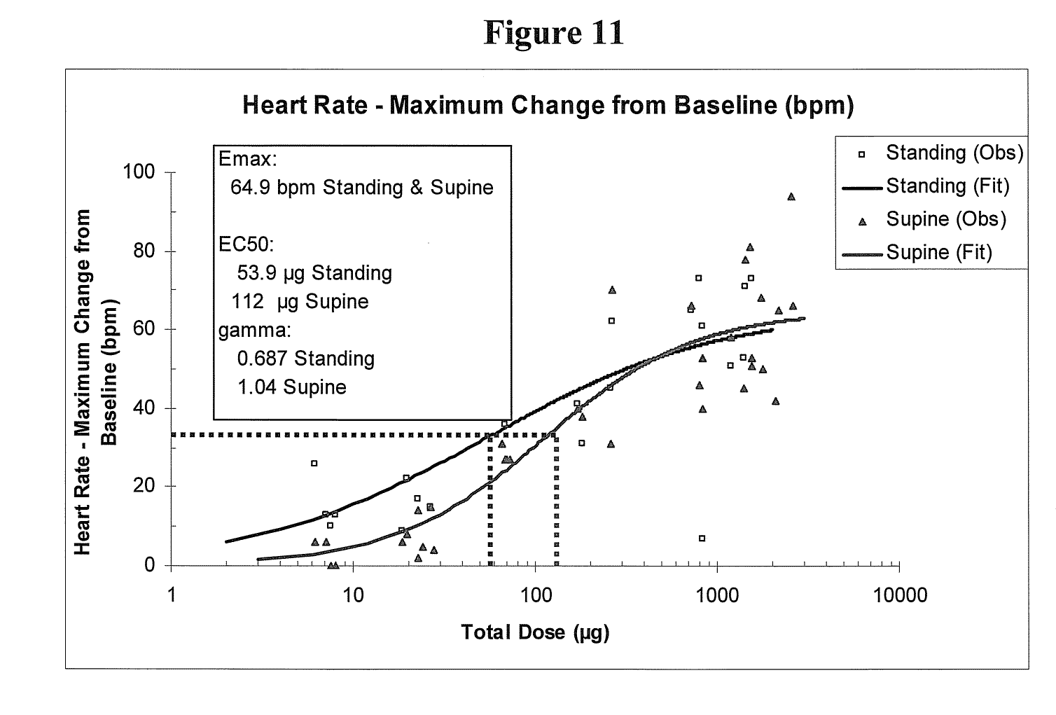

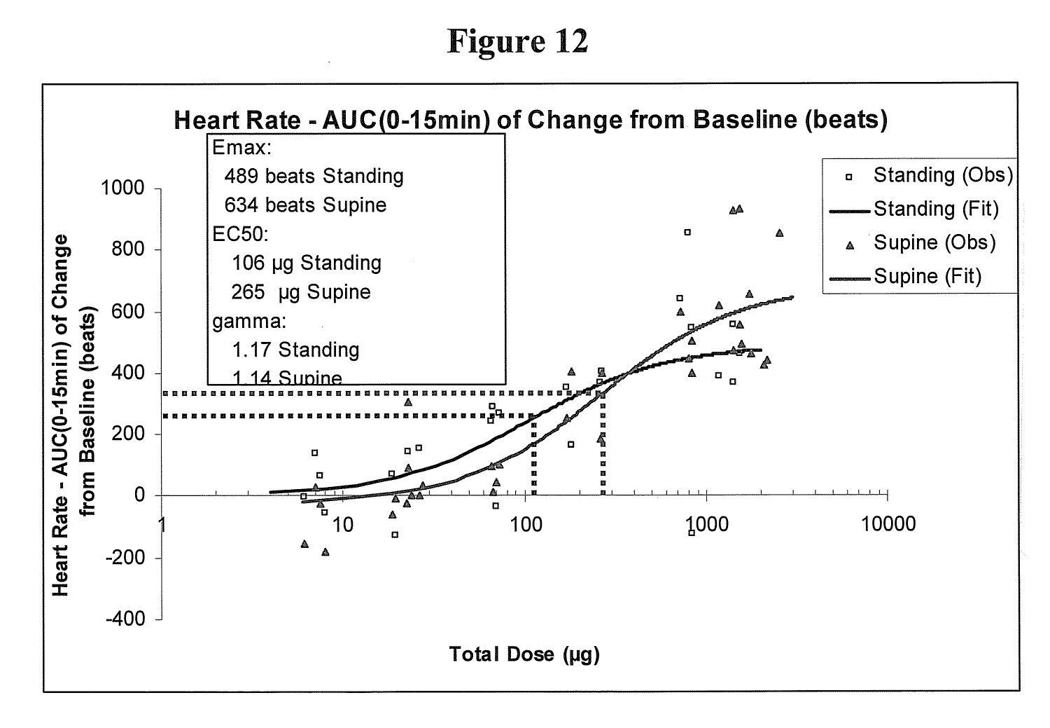

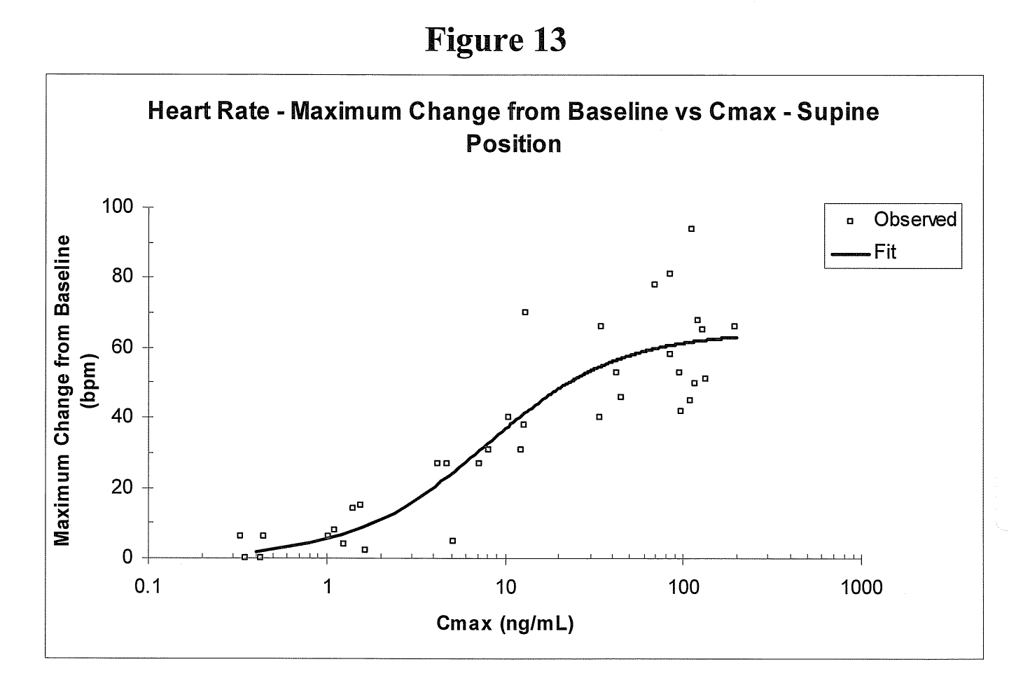

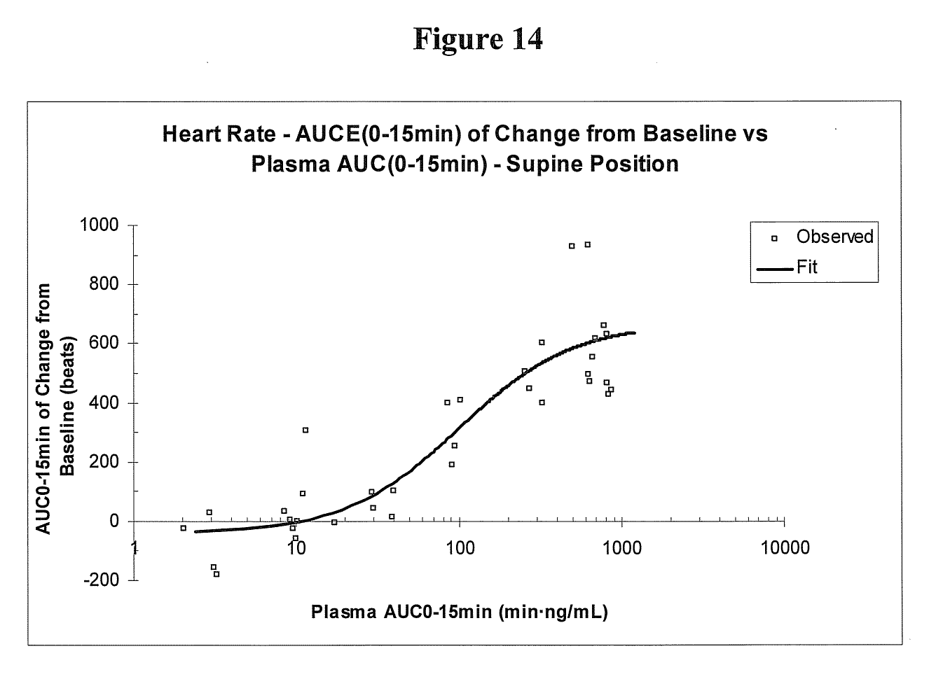

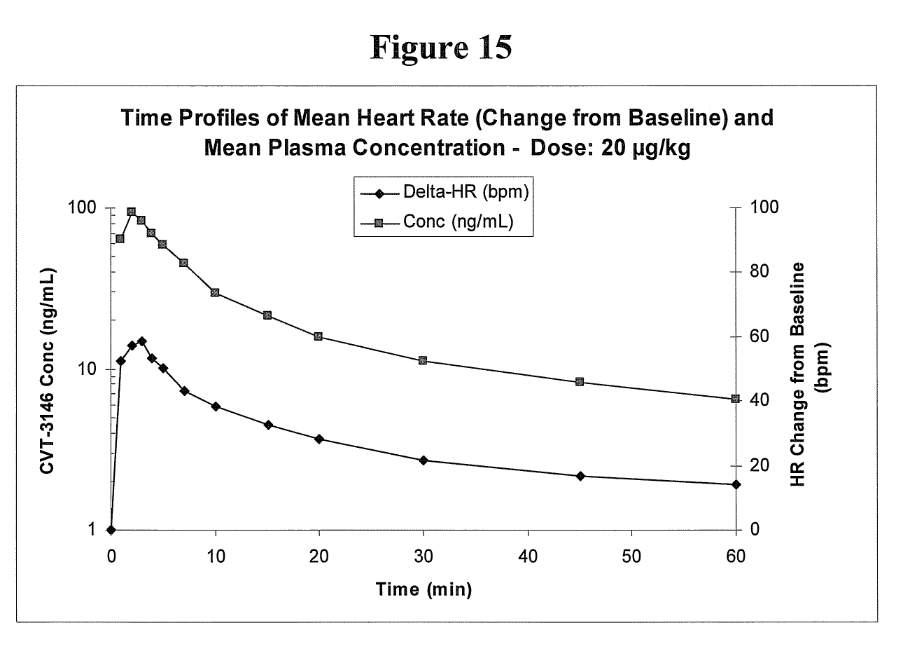

The study evaluated the relationship of changes in heart rate to dose in both standing and supine positions and plasma concentration in the supine position. Some of the study results are reported in FIGS. 8-15.

### Results

### Safety

In general, adverse events reflected the pharmacologic effect of Regadenason and were related to vasodilation or an increase in heart rate (HR). Overall, adverse events were short-lived and mild to moderate in severity. There were no serious adverse events.

Three events were assessed as severe in intensity. (Table 1).

TABLE 1
Adverse Events labeled as severe in intensity

Number of Subjects with AE

| | 20 μg/kg | 30 μg/kg |
|---|---|---|
| Event | Standing | Supine |
| No subjects per group | 4 | 4 |
| Palpitation | 0 | 2 |
| Dizziness | 1 | 0 |
| Syncope | 1 | 0 |

A three-compartment open model was fit to the data using observed Tₘₐₓ (1-4 minutes) as the duration of a zero-order infusion. Reliable parameter estimates were obtained for dose of 1-30 μg/kg. Parameters are summarized in the following (Table 2):

TABLE 2
Mean (SD) Regadenoson Pharmacokinetic Parameters Estimated Using
a Three - Compartment Model

| Dose (μg/kg) | 1 | 3 | 10 | 20 | 30 | Total |
|---|---|---|---|---|---|---|
| N | 3 | 4 | 4 | 8 | 3 | 22 |
| CL (mL/min) | 737 (106) | 668 (167) | 841 (120) | 743 (123) | 1021 | 768 |
| | | | | | (92.7) | (168) |
| Vc (L) | 9.84 (4.12) | 13.7 (6.06) | 17.9 (6.11) | 12.5 | 15.7 | 13.8 |
| | | | | (5.83) | (4.59) | (5.67) |
| Vss (L) | 69.0 (28.2) | 90.0 (29.6) | 101 (11.3) | 75.2 | 89.6 | 75.5 |
| | | | | (10.6) | (10.9) | (24.4) |
| α Half-life | 2.14 | 3.11 | 4.15 | 4.69 | 3.00 | 3.73 |
| (min) | (1.38) | (2.14) | (2.75) | (4.01) | (1.05) | (2.88) |
| β Half-life | 8.93 | 17.2 | 50.2 | 32.6 | 14.0 | 27.2 |
| (min) | (4.10) | (11.4) | (52.1) | (32.4) | (4.98) | (31.0) |
| λ Half-life | 99.0 | 130 | 132 | 117 | 99.4 | 86.4 |
| (min) | (28.6) | (23.1) | (20.5) | (36.0) | (8.10) | (57.5) |
| K21 (1/min) | 0.246 | 0.203 | 0.187 | 0.387 | 0.0948 | 0.258 |
| | (0.255) | (0.272) | (0.305) | (0.615) | (0.0443) | (0.410) |
| K31 (1/min) | 0.01808 | 0.0152 | 0.0108 | 0.0141 | 0.0148 | 0.0143 |
| | (0.00548) | (0.00490) | (0.00592) | (0.00728) | (0.000900) | (0.00580) |

CL = clearance
Vc = central volume of distribution
Vₛₛ = volume of distribution at steady state
K₂₁ = the rate constant for transfer from first peripheral to central compartment
K₃₁ = rate constant for transfer from second peripheral to central compartment

### Results

Regadenoson was well-tolerated, with AE's mainly representing its pharmacological effects as an A_{2A} adenosine receptor agonist.
Mean tolerable dose for Regadenoson was 10 μg/kg standing and 20 μg/kg supine.
Regadenoson does not require weight-adjusted dosing.
There was no time lag between plasma concentration changes and changes in heart rate.
The relationship between HR increase and dose or concentration was adequately described with a sigmoidal Emax model.

### EXAMPLE 4

Regadenoson is a novel selective A_{2A} adenosine receptor agonist being developed as a pharmacologic stressor for radionuclide myocardial perfusion imaging. Previously it has been shown that Regadenoson causes coronary vasodilation without significantly affecting either total peripheral resistance or renal blood flow in awake dogs. The goal of this study was to determine the differential effects of Regadenoson on blood flow velocity in various vascular beds.

The effect of Regadenoson was studied on the blood flow velocity in left circumflex coronary artery (LCX), brain arterial vasculature (BA), forelimb artery (FA) and pulmonary artery (PA) of comparable diameter in the anesthetized dog. Regadenoson (1.0 μg/kg) was administered as an intravenous bolus, transiently enhanced blood flow which was site specific. The effects of Regadenoson were quantified as the average peak blood flow velocity (APV) using intravascular Doppler transducer tipped catheter. Heart rate (HR) and systemic arterial blood pressure (BP) were also monitored.

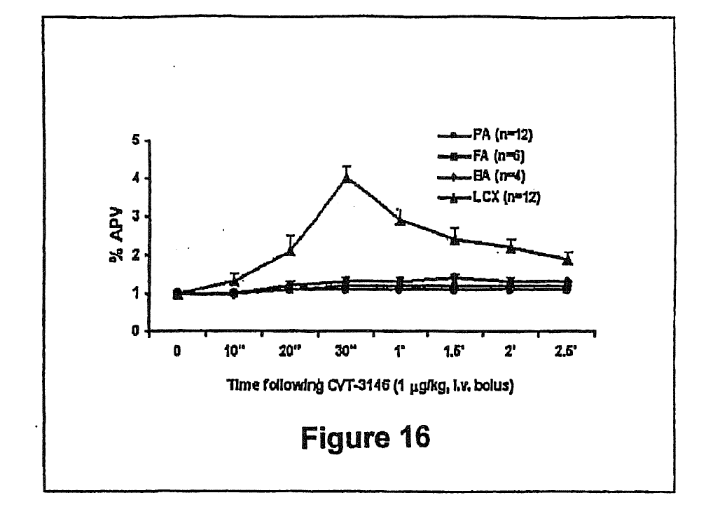

APV increased 3.1±0.2, 1.4±0.1, 1.2±0.1, and 1.1±0.01 fold in the LCX, BA, FA and PA, respectively manifesting a site-potency rank order of LCX>>BA>FA>PA (FIG. 16). The effect of CVT-3146 on blood flow velocity was short lasting; reaching a peak in less than 30 sec and dissipating in less than ten minutes. Increased blood flow velocity was associated with a small transient increase in HR (16 bpm) and decrease in BP (12 mmHg). In conclusion, this study demonstrated that Regadenoson is a potent, short lasting vasodilator that is highly selective for the coronary vasculature.

### EXAMPLE 5

The present study was carried out to determine whether Regadenoson, a selective A_{2A} adenosine receptor agonist, causes sympathoexcitation.

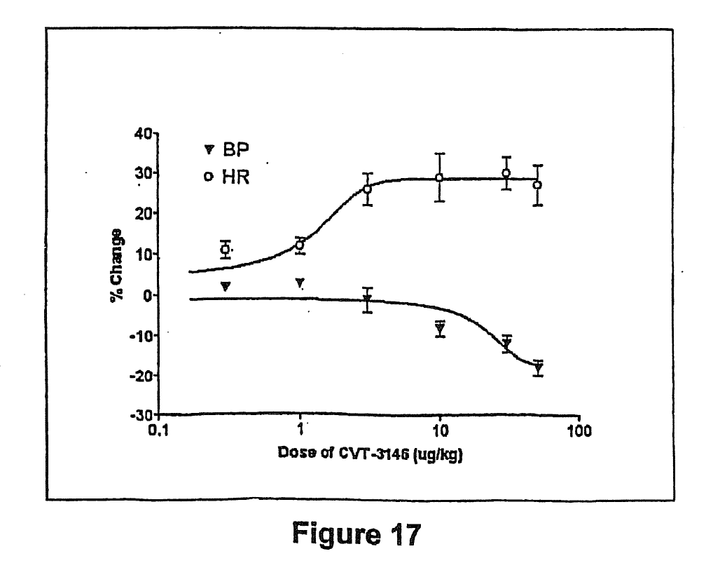

CVT (0.31 μg/kg-50 μg/kg) was given as a rapid i.v. bolus to awake rats and heart rate (HR) and blood pressure (BP) were monitored. Regadenoson caused an increase in BP and systolic pressure (SP) at lower doses while at higher doses there was a decrease in BP and SP. Regadenoson caused a dose-dependent increase in HR (FIG. 17). The increase in HR was evident at the lowest dose of CVT at which there was no appreciable decrease in BP. ZM241385 (30 μg/kg, N=5), an A_{2A} adenosine receptor antagonist, attenuated the decrease in BP (Regadenoson: 14±3%, ZM: 1±1%) and the increase in HR (CVT: 27±3%, ZM: 18±3%) caused by Regadenoson. Pretreatment with metoprolol (MET, 1 mg/kg, n=5), a beta-blocker, attenuated the increase in HR (CVT: 27±3%, MET: 15±2%), but had no effect on hypotension caused by Regadenoson. In the presence of hexamethonium (HEX, 10 mg/kg, n=5), a ganglionic blocker, the tachycardia was prevented (CVT: 27±3%, HEX: −1±2%), but BP was further reduced (CVT: −11±2%, HEX: −49±5%). Regadenoson (10 μg/kg, n=6) also significantly (p<0.05) increased plasma norepinephrine (control: 146±11, Regadenoson 269±22 ng/ml) and epinephrine (control:25:f:5, CVT:I00:f:20 ng/ml) levels. The separation of HR and BP effects by dose, time and pharmacological interventions provides evidence that tachycardia caused by Regadenoson is independent of the decrease in BP, suggesting that Regadenoson, via activation of A_{2A} adenosine receptors may cause a direct stimulation of the sympathetic nervous system.

### EXAMPLE 6

Pharmacologic stress SPECT myocardial perfusion imaging (MPI) with adenosine (A) is a well-accepted technique, with excellent diagnostic and prognostic value and proven safety. However, side effects are common and AV nodal block and severe flushing are poorly tolerated. Agents such as Regadenoson selectively act upon the A_{2A} adenosine receptor and avoid stimulation of other receptor subtypes which may prevent such adverse reactions.

To determine the ability of Regadenoson to produce coronary hyperemia and accurately detect CAD, 35 subjects (26 men, 9 women; 67±10 years) underwent both A and Regadenoson stress/rest MPI, with 10.0±9.1 days between studies. Prior MI was noted in 12 patients, and many had prior revascularization [CABG (n=19), PCI (n=22)]. Regadenoson [400 mcg (n=18), 500 mcg (n=17)] was administered as an IV bolus immediately followed by a saline flush, and then a Tc-99m radiopharmaceutical [sestamibi (n=34), tetrofosmin (n=1)]. SPECT images were uniformly processed, intermixed with control studies (normal and fixed-only defects), and interpreted by three observers in a blinded fashion using a 17-segment model. Quantitative analysis was also performed using 4D MSPECT. In addition to three separate readings, a consensus interpretation was performed and then a direct, same-screen comparison of A and REGADENASON images undertaken to determine relative differences, using 5 regions per study.

The summed scores following stress were similar, both with visual (A=133.9±1.5, Regadenoson=13.2±1.3; p=n.s.) and quantitative methods of analysis (A=13.7±1.5, Regadenoson=133.6±1.6; p=n.s.). Similarly, comparisons between the summed rest and summed difference scores were identical. The direct comparison also revealed no differences in ischemia detection, with a regional concordance for ischemia extent and severity of 86.3% and 83.4%, respectively. No dose-dependent effect of Regadenoson on ischemia detection was noted. A conclusion of the study is that Regadenoson, administered by a logistically simple bolus injection, provides a similar ability to detect and quantify myocardial ischemia with SPECT MPI as noted with an A infusion.

### EXAMPLE 7

Regadenoson is a selective A_{2A} adenosine receptor agonist that produces coronary hyperemia and potentially less adverse effects due to its limited stimulation of receptor subtypes not involved with coronary vasodilation. This study evaluated the effectiveness of Regadenoson as a pharmacologic stress agent.

36 subjects (27 men, 9 women; 67±10 years) were studied with two doses of Regadenoson [400 mcg (n=18), 500 mcg (n=18)], administered as an IV bolus, as part of a pharmacologic stress myocardial perfusion imaging protocol.

Adverse effects (AE) occurred in 26 pts (72%), including chest discomfort (33%), headache (25%), and abdominal pain (11%), with a similar incidence for both doses. Flushing, dyspnea, and dizziness were more frequent in the 500-mcg group (44%, 44%, and 28%, respectively) than in the 400-mcg group (17%, 17%, and 11%, respectively). Most AEs were mild to moderate (96%) and resolved within 15 min without treatment (91%). One serious AE occurred, with exacerbation of a migraine headache, which required hospitalization. ST and T wave abnormalities developed with Regadenoson in 7 and 5 pts, respectively. No 2nd or 3rd degree AV block was noted and there were no serious arrhythmias. Peak hemodynamic effects are shown in Table 3 and were noted at 4 min for systolic blood pressure (BP), 8 min for diastolic BP, and within 2 min for heart rate (HR). The effect on BP was minimal and systolic BP did not fall below 90 mmHg with either dose. The mean change in HR response was higher for the 500 mcg dose (44.2%) than for 400 mcg (34.8%; p=n.s.). Thirty min after Regadenoson, BP changes deviated <2% from baseline but HR remained above baseline by 8.6%.

The results of this study indicate that Regadenoson is well-tolerated and has acceptable hemodynamic effects. Minimal differences were noted in BP and HR responses between the 400 mcg and 500 mcg doses, but AEs were more frequently at the higher dose. Regadenoson appears safe and well-tolerated for bolus-mediated pharmacologic stress perfusion imaging. Hemodynamic Changes (mean ±S.D.)

TABLE 3

| | Absolute Change | Relative Change |
|---|---|---|
| Heart Rate | +21.9 ± 10.4 beats per min | +36.7% + 21.0% |
| Systolic BP | −5.9 ± 10.7 mmHg | −4.1% ± 7.6% |
| Diastolic BP | −5.4 ± 7.2 mmHg | −7.9% ± 10.5% |

### EXAMPLE 8

In this study the vasodilator effects of Regadenoson were compared to those of ADO in different vascular beds in awake dogs. Dogs were chronically instrumented for measurements of the blood flow in coronary (CBF), mesenteric (MBF), hind limb (LBF), and renal (RBF) vascular beds, and hemodynamics. Bolus injections (iv) to Regadenoson (0.1 to 2.5 μg/kg) and ADO (10 to 250 μg/kg) caused significant increases in CBF (35±6 to 205±23% and 58±13 to 163±16%) and MBF (18±4 to 88±14% and 36±8 to 84±5%).

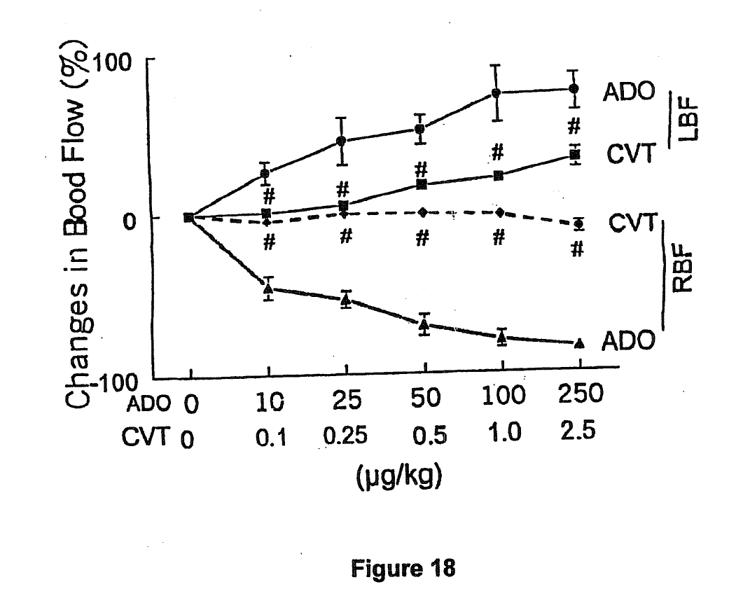

The results of the study demonstrate that Regadenoson is a more potent and longer lasting coronary vasodilator compared to ADO (the duration for CBF above 2-fold of the baseline; Regadenoson (2.5 μg/kg): 130±19s; ADO (250 μg/kg): 16±3s, P<0.5). As shown in FIG. 18 (mean ±SE, n=6), Regadenoson caused a smaller increase in LBF than ADO. ADO caused a dose-dependent renal vasoconstriction (RBF −46±7 to −85±4%), whereas Regadenoson has no or a little effect on RBF (−5±2 to −11±4%, P<0.05, compared to ADO). In conclusion, Regadenoson is a more selective and potent coronary vasodilator than ADO. Regadenoson has no the significant effect on renal blood flow in awake dogs. These features of Regadenoson make it an ideal candidate for radionuclide myocardial perfusion imaging.

### EXAMPLE 9

A Randomized Double-Blind, Placebo-Controlled, Cross-Over Study to Evaluate the Effect of Regadenoson on Pulmonary Function in AMP-Sensitive Subjects with Mild or Moderate Asthma

This was a double-blind, Phase 2, cross-over study designed to evaluate whether Regadenoson at a dose to be used for myocardial perfusion imaging in the detection of coronary artery disease (400 μg) elicited a bronchoconstrictive response in subjects with mild or moderate asthma who showed a reduction in forced expiratory volume over 1 second (FEV₁) of at least 20% with a standard AMP challenge at screening.

The primary objective was to compare the incidence of bronchoconstrictive reactions, defined as reduction from baseline in FEV₁ of >15% within 2 hours following an intravenous (iv) bolus of 400 micrograms of Regadenoson or matching placebo.

Men or women ≧18 years of age with a diagnosis of mild or moderate asthma as documented by clinical history and pulmonary function test were considered for inclusion. The target was to enrolled 48 evaluable subjects: 24 mild and 24 moderate asthma subjects. Mild asthma subjects must not have had corticosteroids (inhaled or oral) within 8 weeks prior to the screening visit and must have had an FEV₁≦80% of the predicted value at screening. Moderate asthma subjects may have been taking corticosteroids and must have had an FEV₁>60% and <80% of the predicted value at screening. Patients were not permitted short-acting bronchodilators for >6 hours and long-acting bronchodilators and methylxanthines for >24 hours prior to AMP, Regadenoson, or placebo.

When 24 mild asthma subjects had completed the study, an independent safety review was conducted based on predetermined clinical criteria before initiating enrollment of moderate asthma subjects.

### AMP Challenge

AMP (adenosine 5′-monophosphate sodium salt) was used as an indirect bronchoconstrictor stimulus to select a group of susceptible subjects with adenosine-mediated bronchial hyperreactivity. A standard clinical protocol utilized by the investigative site for the inhalation of AMP for the purpose of provoking a bronchoconstrictor response in the airways was adopted for screening of all subjects. On arrival in the unit, subjects were required to rest for 15 minutes before assessment of lung function at baseline, measured as the best of 3 technically acceptable recordings of FEV₁ taken at 1-minute intervals. Subject inhaled a series of five breaths of saline as control, followed by a breath of each increasing concentration of AMP at 3-minute intervals. Two measurements of FEV₁ were to be made 90 and 150 seconds after each saline inhalation. The highest FEV₁ value was to be recorded. Unless a fall in FEV₁ of >10% from the baseline value was observed, subjects then inhaled increasing doubling concentrations of AMP (starting at 0.39 mg/mL) until a ≧20% decrease of FEV₁ from the post-saline value was recorded. FEV₁ was to be recorded at 90 and 150 seconds after each concentration was administered. Subjects were qualified if they had demonstrated a PC20 to AMP <400 mg/mL.

### Drug Administration

Subjects received 400 μg Regadenoson or placebo administered as an intravenous (iv) bolus in a double-blind cross-over design. Repeated measurements of FEV₁ as an assessment of bronchoconstriction were performed before and for up to 2 hours after study drug administration.

### Results

The mean age (±SD) of the patients was 27 i 6 years and 65% were male. The mean baseline FEV₁ in the mild (n=24) and moderate (n=24) groups were 3.88±0.81 L and 2.77±0.64 L, respectively.

None of the subjects with mild asthma had bronchoconstriction following placebo or Regadenoson. A total of 4 moderate asthma subjects had bronchoconstrictive reactions. There was no statistically significant difference between the number of moderate asthma subjects having bronchoconstriction while taking placebo (n=2) or Regadenoson (n=2) (p=0.99). These 4 subjects had decreases in FEV₁ ranging from 7-12% following treatment in the cross-over arm that were no considered clinically significant. None of these patients experienced a serious adverse event or a pulmonary adverse event, or prematurely terminated from the study. The greatest decrease in FEV₁ (36%) occurred in 1 patient at 90 minutes following Regadenoson.

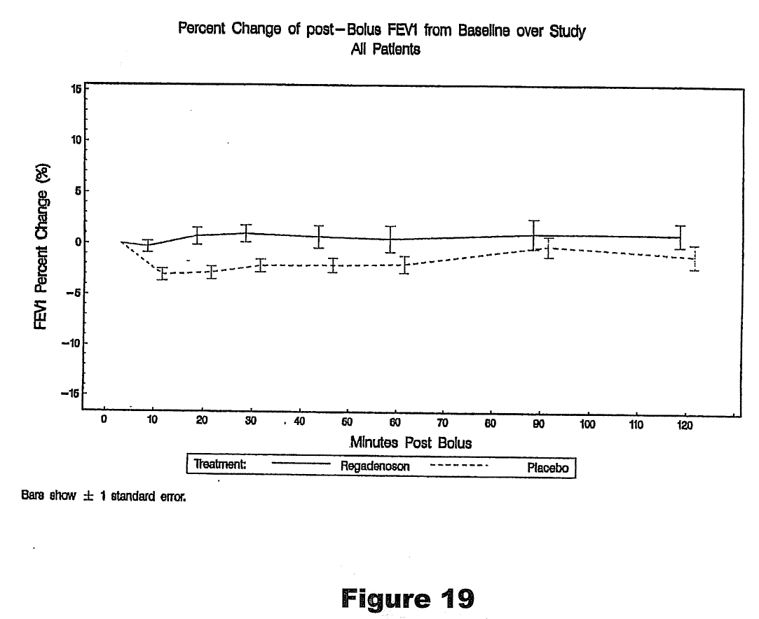

The ratio of post-bolus FEV₁ to baseline FEV₁ was calculated for each of the 7 time points after study drug administration. In addition, the ratio of lowest post-bolus FEV₁ to baseline FEV₁ was also assessed. There were no clinical meaningful differences between regadenoson and placebo in these parameters. (See FIG. 19.)

At the follow-up physical (2 hours), no patient had abnormalities. More adverse events occurred after Regadenoson compared with placebo (98% vs. 8%). The most common adverse events included tachycardia (66%), dizziness (53%), headache (45%), dyspnea (34%), flushing (32%), chest discomfort (21%), nausea (19%), and paraesthesia (19%).

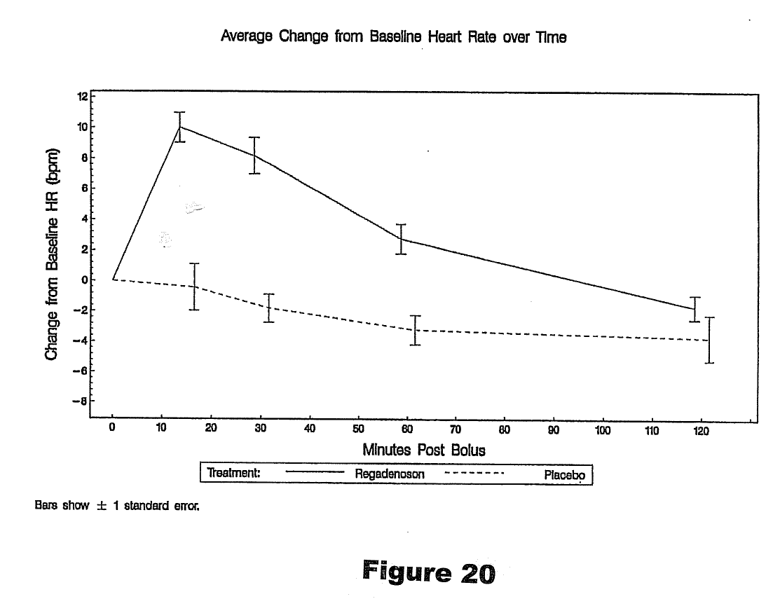

Regadenoson significantly increased HR (maximum of +10.4 bpm) compared with the placebo treatment. This increase was still evident 30 and 60 minutes after dosing with regadenoson. HR returned to within 5 bpm of baseline by 60 minutes post-regadenoson. (See FIG. 20.)

### Conclusions

There was no demonstrable difference between Regadenoson and placebo in the mean FEV₁ or incidence of bronchoconstrictive reactions in susceptible asthma patients, although one patient had a substantial FEV₁ reduction (−36%) after Regadenoson administration. The significant increase in heart rate and adverse events associated with Regadenoson were consistent with its pharmacologic action.

### EXAMPLE 10

### Background

Because of its non-selective adrenoreceptor agonist activity, adenosine (ADO) may aggravate cardio-receptor symptoms in patients with chronic obstructive pulmonary disease (COPD). It was hypothesize that as Regadenoson selectively activities the A_{2A}-adenosine receptor in the coronary circulation, it may be better tolerated in COPD patients compared to ADO.

### Methods and Results:

COPD patients were selected from two phase III randomized, triple-blind, placebo-controlled, multicenter studies (N=2,015) designed to test the strength of agreement for reversible cardiac defects between ADO and REG. There were 35 patients in the ADO group and 69 in the Regadenoson group. Age and gender were similar between ADO (68=11 yrs; 77% male) and Regadenoson group (68±11 yrs; 81% male). Of the 35 patients, 5 suffered (14%) cardiac symptoms in the ADO group vs 3/69 (4%) in the Regadenoson group (p=0.12); 14/35 (40%) in the ADO vs. 20/69 (29%) in the Regadenoson group resorted respiratory symptoms (p=0.28). Angina, occurrence of second degree AV block, acute pulmonary oedema and ronchi were higher in ADO group; nausea, GI discomfort and headache occurred more often in the Regadenoson group.

### Conclusion:

Compared to ADO, patients with COPD appeared to have fewer cardio-pulmonary side-affects following administration of Regadenoson. Thus, Regadenoson demonstrated a more favorable safety profile when used as a stress agent in patients with COPD in this study.

### EXAMPLE 11

### Background

Patients with reactive airways are at risk for adenosine-induced bronchoconstriction, mediated via A_{2B} and/or A₃ adenosine receptors. The following study assess whether regadenoson (REG), an agent being developed for myocardial perfusion, would or would not elicit bronchospasm in susceptible patients because it is a selective A_{2A} adenosine receptor agonist.

### Methods:

Two similar randomized, double-blind, placebo (PLC)-controlled crossover trials were conducted, one in asthmatics with a positive adenosine monophosphate (AMP) challenge (a validated marker of airway inflammation) and one in patients with moderate or several chronic obstructive pulmonary disease (COPD). In both studies, short-acting bronchodilators were held prior to and for the 120 minute time frame study drug treatment during which spirometry was repeatedly assessed.

### Results:

The mean ages and baseline FEV1 values of the asthma and COPD study patients were 27 (6) and 67 (11.9) and 3.33 (0.91)L and 1.58 (0.57)L, respectively. The nature of adverse subjects following REG in either study were: tachycardia, dizziness, headache, dyspnea, flushing, chest discomfort, parasthesia, and nausea. Dyspnea occurred commonly following REG treatment (34% and 61% in the asthma and COPD studies, respectively), but did not correlate with FEV1 in either study. See Table 4 below for additional data.

TABLE 4

| | ASTHMA | |
|---|---|---|
| | STUDY | COPD STUDY |
| ENDPOINT | (N = 48) | (N = 49) |
| Mean change from baseline in | p = 0.17 for all | p > 0.2 for the |
| FEV1 or Mean FEV1 at all post- | post-bolus time | change at all post- |
| bolus time points following REG | points combined: | bolus time points |
| vs. PLC | mean FEV1 | |
| | 3.33 (0.9)L REG | |
| | vs. | |
| | 3.27 (0.91)L PLC | |
| Maximum decline in FEV1 | Not Done | 0.11 (0.14)L REG |
| following REG vs. PLC | | vs. |
| | | 0.12 (0.10)L PLC |
| | | (p = 0.55) |
| Maximum decline in oxygen | Not Done | 1.21 (1.30)% REG |
| saturation following REG vs. | | vs. |
| PLC | | 1.12 (1.43)% PLC |
| | | (p = 0.72) |
| Number of patients with new | Not Done | 3 REG, 6 PLC |
| onset wheezing following REG | | (p = 0.33) |
| vs. PLC | | |
| Number of patients with use of | 0 REG, 0 PLC | 0 REG, 0 PLC |
| acute oxygen or short-acting | | |
| bronchodilators | | |
| Number of patients with FEV1 | 2 REG, 2 PLC | 6 REG, 3 PLC |
| decline by >15% over 120 min | (p = 0.99) | (p = 0.31) |
| Number of patients with FEV1 | 1 REG, 0 PLC | 2 REG, 3 PLC |
| decline by >20% over 120 min | | |
| Number of patients with FEV1 | 1 REG, 0 PLC | 0 REG, 1 PLC |
| decline by >30% over 120 min | | |

### Conclusions:

There were few demonstrable difference between REG and PLC across a multitude of pulmonary assessments in 2 controlled trials of susceptible patients with reactive airways.

We claim:

1. A method of diagnosing myocardial dysfunction during vasodilator induced myocardial stress perfusion imaging in a human patient having a history of pulmonary disease, comprising administering at least 10 μg of at least one partial A_{2A} adenosine receptor agonist to the mammal.

2. The method of claim 1, wherein no more than about 1000 μg of the partial A_{2A} adenosine receptor agonist is administered to the mammal.

3. The method of claim 1, wherein the amount of the partial A_{2A} adenosine receptor agonist administered is greater than about 600 μg.

4. The method of claim 1, wherein the amount of the partial A_{2A} adenosine receptor agonist administered is greater than about 100 μg.

5. The method of claim 1, wherein the amount of the partial A_{2A} adenosine receptor agonist administered ranges from about 10 to about 600 μg.

6. The method of claim 5, wherein the A_{2A} adenosine receptor is administered in a single dose.

7. The method of claim 6, wherein the partial A_{2A} adenosine receptor agonist is administered by iv bolus.

8. The method of claim 6, the partial A_{2A} adenosine receptor agonist is administered in less than about 10 seconds.

9. The method of claim 6, wherein the amount of the partial A_{2A} adenosine receptor agonist administered is greater than about 500 μg.

10. The method of claim 6, wherein the partial A_{2A} adenosine receptor agonist is administered in an amount ranging from about 100 μg to about 500 μg.

11. The method of claim 1, wherein the partial A_{2A} adenosine receptor agonist is selected from the group consisting of CVT-3033, Regadenoson, and combinations thereof.

12. The method of claim 6, wherein the partial A_{2A} adenosine receptor agonist is Regadenoson.

13. A method of diagnosing myocardial dysfunction during vasodilator induced myocardial stress perfusion imaging in a human patient having a history of pulmonary disease, comprising administering a radionuclide and a partial A_{2A} receptor agonist in an amount ranging from about 10 to about 600 μg wherein the myocardium is examined for areas of insufficient blood flow following administration of the radionuclide and the partial A_{2A} receptor agonist.

14. The method of claim 13, wherein the myocardium examination begins within about 1 minute from the time the partial A_{2A} adenosine receptor agonist is administered.

15. The method of claim 13, wherein the administration of the partial A_{2A} adenosine receptor agonist causes at least a 2.5 fold increase in coronary blood flow.

16. The method of claim 15, wherein the at least a 2.5 fold increase in coronary blood flow that is achieved within about 1 minute from the administration of the partial A_{2A} adenosine receptor agonist.

17. The method of claim 13, wherein the radionuclide and the partial A_{2A} adenosine receptor agonist are administered separately.

18. The method of claim 13, wherein the radionuclide and the partial A_{2A} adenosine receptor agonist are administered simultaneously.

19. The method of claim 15, wherein the at least a 2.5 fold increase in coronary blood flow is less than about 5 minutes in duration.

20. The method of claim 19, wherein the at least a 2.5 fold increase in coronary blood flow is less than about 3 minutes in duration.

21. The method of claim 13, wherein the partial A_{2A} adenosine receptor agonist is Regadenoson.

22. A method of diagnosing myocardial dysfunction during vasodilator induced myocardial stress perfusion imaging in a human patient having a history of pulmonary disease, comprising administrating Regadenoson in an amount ranging from about 10 to about 600 μg in a single iv bolus.

23. A method of myocardial dysfunction during vasodilator induced myocardial stress perfusion in a human patient having a history of pulmonary disease, comprising administrating Regadenoson in an amount ranging from about 100 to about 500 μg in a single iv bolus.
